(19) United States
(12) Patent Application Publication
(10) Pub. No.: US 2023/0357314 A1
(43) Pub. Date: Nov. 9, 2023

(54) METHODS FOR ULTRAFAST FORMATION OF DISULFIDE BONDS IN PEPTIDES AND PROTEINS

(71) Applicant: TECHNION RESEARCH & DEVELOPMENT FOUNDATION LIMITED, Haifa (IL)

(72) Inventors: Ashraf BRIK, Haifa (IL); Shay LAPS, Haifa (IL); Fatima ATAMLEH, Haifa (IL); Guy KAMNESKY, Haifa (IL)

(21) Appl. No.: 18/028,179

(22) PCT Filed: Sep. 23, 2021
(86) PCT No.: PCT/IL2021/051161

§ 371 Date: Mar. 23, 2023

Related U.S. Application Data
(60) Provisional application No. 63/082,515, filed on Sep. 24, 2020.
(60) Provisional application No. 63/216,050, filed on Jun. 29, 2021.

(51) Int. Cl. C07K 1/113; C07K 1/06

(52) U.S. Cl. CPC C07K 1/1133; C07K 1/067

(57) ABSTRACT

Provided herein a method for forming one or more intramolecular disulfide bonds.

### CROSS REFERENCE TO RELATED APPLICATIONS

This application claims the benefit of priority under 35 U.S.C. § 119(e) of U.S. Provisional Patent Application Nos. 63/082,515, filed on Sep. 24, 2020, and 63/216,050, filed on Jun. 29, 2021. The contents of the above applications are all incorporated by reference as if fully set forth herein in their entirety.

### FIELD OF THE INVENTION

The present invention is in the field of synthetic reactions.

### BACKGROUND OF THE INVENTION

After production in the ribosome, most proteins undergo further maturation through covalent modifications, which alter their structure, localization or function and aberrations in these steps are associated with numerous diseases. While unmodified proteins can be readily obtained by biological expression, the preparation of posttranslationally modified proteins remains challenging, increasing demand for their chemical synthesis.

Disulfide bonds, one of the most widespread covalent modifications, influences the three-dimensional architecture and function of peptides and proteins exist in many target of therapeutic interest such as in the insulin hormone, the coronavirus (SARS-CoV-2) spike protein, and in natural libraries of toxin peptides among others. Unfortunately, performing studies with isolated or biologically expressed peptides and proteins that contain disulfide bonds, is time-consuming and often impractical and even impossible for various targets, driving the great interest in finding approaches for their efficient synthesis. Yet, their chemical synthesis remains an unsolved problem for over six decades, leaving millions of potential therapeutic targets inaccessible.

Therefore, a need exists in the field of peptide synthesis to search for simple and efficient methods of post-synthetic disulfide bond formation.

### SUMMARY OF THE INVENTION

In one aspect of the invention, there is provided a method for forming a plurality of disulfide bonds, comprising a. providing a peptide comprising (i) a deprotected cysteine pair, (ii) a first cysteine pair protected by a first protecting group, and optionally (iii) a second cysteine pair protected by a second protecting group; b. adding to the peptide a sufficient amount of a reagent comprising a disulfide, a diselenide, a thiol, a sulfide, a thiocarbonate, a dithiocarbonate, thiocarbamate, or a dithiocarbamate, including any combination, a disulfanyl derivate, a metal complex or a salt thereof, thereby forming a first disulfide bond between thiols of the deprotected cysteine pair; c. providing the peptide under conditions sufficient for deprotecting the first protecting group and adding a sufficient amount of the reagent to the peptide, thereby forming a second disulfide bond between thiols of the first cysteine pair; wherein the plurality of disulfide bonds comprises intramolecular bonds; and wherein the steps b and c are performed in one pot.

In some embodiments, the step b is performed prior to the step c or subsequent to the step c.

In some embodiments, the method further comprises step d, comprising adding a sufficient amount of the reagent to the peptide, or to a reaction mixture comprising the peptide; and providing the peptide or the reaction mixture under conditions sufficient for deprotecting the second protecting group, thereby forming a second disulfide bond between thiols of the second cysteine pair.

In some embodiments, the step d is performed prior to the step c or subsequent to the step c.

In some embodiments, the steps b to d are performed in one pot.

In some embodiments, any one of the first protecting group and the second protecting group independently comprises a photocleavable protecting group or a photostable protecting group.

In some embodiments, the photocleavable protecting group comprises o-nitrobenzyl (NBzl).

In some embodiments, the conditions sufficient for deprotecting the photostable protecting group comprise adding a sufficient amount of a transition metal catalyst to the peptide.

In some embodiments, the transition metal catalyst comprises a transition metal, a complex or a salt thereof.

In some embodiments, the transition metal comprises a metal selected from the group consisting of Pt, Pd, Ru, Cu, Ni, Co, Ti, Zn and Ag or any combination thereof, and wherein the metal is in an elemental state or in an oxidized state.

In some embodiments, the transition metal catalyst comprises Pd, including any salt or a complex thereof.

In some embodiments, the photostable protecting group comprises acetamidomethyl.

In some embodiments, the method further comprises adding an effective amount of a complexing agent to the peptide in contact with the transition metal catalyst.

In some embodiments, the effective amount of the complexing agent is sufficient for substantially complexing the transition metal catalyst.

In some embodiments, the complexing agent comprises at least one of a thiol, a thiocarbonate, a dithiocarbonate, thiocarbamate, or a dithiocarbamate, including any disulfanyl derivate thereof.

In some embodiments, the complexing agent comprises glutathione.

In some embodiments, the conditions sufficient for deprotecting the photocleavable protecting group comprises exposing the peptide to light for a time period sufficient for de-protection of the photocleavable protecting group.

In some embodiments, the light has a wavelength suitable for deprotecting the photocleavable protecting group.

In some embodiments, the light is characterized by a wavelength of between 200 and 600 nm.

In some embodiments, the reagent is selected from the group consisting of Pd, Cu, sulfide, glutathione, disulfiram (DSF), and diethyldithiocarbamate (DTC), including any combination, any complex or a salt thereof.

In another aspect of the invention, there is provided a method for forming a disulfide bond, comprising: a. providing a peptide comprising a first cysteine pair, wherein each thiol of the first cysteine pair is protected by a photocleavable protecting group; b. exposing the peptide to light for a time period sufficient for substantially deprotecting the photocleavable protecting group; c. adding a sufficient amount of a reagent comprising a disulfide, a diselenide, a thiol, a sulfide, a thiocarbonate, a dithiocarbonate, thiocarbamate, or a dithiocarbamate, including any combination, a disulfanyl derivate, a metal complex or a salt thereof, thereby forming a first disulfide bond between thiols of the first cysteine pair; wherein the disulfide bond is an intramolecular bond; and wherein the step b and the step c are performed simultaneously or subsequently.

In some embodiments, the peptide further comprises an additional cysteine pair, wherein the additional cysteine pair is protected by a photostable protecting group.

In some embodiments, the method further comprises step d of (i) providing the peptide under conditions sufficient for substantially deprotecting the photostable protecting group; and (ii) adding a sufficient amount of the reagent; thereby forming a second disulfide bond between thiols of the additional cysteine pair.

In some embodiments, the conditions sufficient for deprotecting the photostable protecting group comprise adding a sufficient amount of a transition metal catalyst to the peptide.

In some embodiments, the method further comprises adding an effective amount of a complexing agent to the peptide in contact with the transition metal catalyst.

In some embodiments, the effective amount of the complexing agent is sufficient for substantially complexing the transition metal catalyst.

In some embodiments, the complexing agent comprises at least one of a thiol, a thiocarbonate, a dithiocarbonate, thiocarbamate, or a dithiocarbamate, including any disulfanyl derivate thereof.

In some embodiments, the complexing agent comprises glutathione.

In some embodiments, the transition metal catalyst comprises a transition metal, a complex or a salt thereof.

In some embodiments, the transition metal is selected from the group consisting of Pt, Pd, Ru, Cu, Ni, Co, Ti, Zn and Ag or any combination thereof, optionally wherein the transition metal catalyst comprises Pd, a salt or a complex thereof.

In some embodiments, the light has a wavelength suitable for deprotecting the photocleavable protecting group.

In some embodiments, the light is characterized by a wavelength of between 200 and 600 nm.

In some embodiments, the photocleavable protecting group comprises o-nitrobenzyl (NBzl).

In some embodiments, the reagent is selected from the group consisting of Pd, Cu, sulfide, glutathione, disulfiram (DSF), and diethyldithiocarbamate (DTC), including any combination, any complex or a salt thereof.

In some embodiments, the step b is performed prior to the step c; or wherein the step b and the step c are performed simultaneously.

In some embodiments, the step d is performed prior to the steps b and c or subsequent to the steps b and c.

In some embodiments, at least two of the steps a to d are performed in one pot.

In another aspect of the invention, there is provided a composition comprising a peptide comprising one or more intramolecular disulfide bond, and a residual amount of a reagent comprising a disulfide, a diselenide, a thiol, a sulfide, a thiocarbonate, a dithiocarbonate, thiocarbamate, or a dithiocarbamate, including any combination, a disulfanyl derivate, a metal complex or a salt thereof.

In some embodiments, the reagent comprises any of glutathione, disulfiram (DSF), and diethyldithiocarbamate (DTC), including any combination thereof.

In some embodiments, the peptide is synthesized according to the method of the present invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-D Present schemes with the position of modifications in the analogues of Conotoxin mr3e (FIG. 1A), EETI-II (FIG. 1B), linaclotide (FIG. 1C) and plectasin (FIG. 1D);

FIGS. 2A-B present HPLC and mass analyses of Fmoc-solid phase peptide synthesis (SPPS) of α-conotoxin SI bearing NBzl PGs at Cys (2&7) and Acm PGs at Cys (3&13): crude (FIG. 2A): the main peak corresponds to the desired peptide; and purified (FIG. 2B): the main peak corresponds to the desired peptide, with the observed mass 1769.4±0.2 Da, calcd: 1769.6 Da (average isotopes);

FIGS. 3A-F present a scheme of f α-conotoxin synthesis (FIG. 3A) and HPLC and mass analyses of the intermediates in α-conotoxin synthesis: conotoxin peptide bearing Cys (Acm) at positions 3&13 and Cys (NBzl) at positions 2&7 (FIG. 3A, 1 and FIG. 3B), dissolved in 6 M GnHCl, pH 7. At time zero: observed mass: 1769.4±0.1 Da, calcd: 1769.6 Da (average isotopes). Treatment with 15 equiv. PdCl₂ followed by treatment with 30 equiv. DTC and 10 equiv. DSF (FIG. 3A, 2 and FIG. 3C), after 5 min: the main peak corresponds to 1^{st} disulfide formation with the observed mass: 1624.5±0.1 Da, calcd: 1625.6 Da (average isotopes). Exposure to UV (350 nm) at room temperature (FIG. 3A, 3 and FIG. 3D), after 8 min: the main peak corresponds to 1^{st} disulfide formation with the observed mass: 1497.2±0.1 Da, calcd: 1497.6 Da (average isotopes). 2^{nd} Disulfide formation (FIG. 3A, 4 and FIG. 3E) with the observed mass: 1352.5±0.1 Da, calcd: 1353.4 Da (average isotopes); and circular dichroism (CD) graph for synthetic α-conotoxin SI (FIG. 3F);

FIGS. 4A-E present a schematic representation of the syntheses of the six EETI-II analogues (FIG. 4A) via treatment with DSF for 10 s, then UV (350 nm) for 8 min and Pd^{II}/DTC+DSF for 5 min, and HPLC comparison of R₁=H, R₂=Acm, R₃=NBzl (FIG. 4B, 1), R₁=H; R₂=NBzl, R₃=Acm (FIG. 4B, 2), the main peak corresponds to fully oxidized EETI-II with the observed mass: 2879.2±0.1 Da, calcd: 2879.4 Da (average isotopes); R₁=NBzl, R₂═H, R₃=Acm (FIG. 4B, 3); R₁=NBzl, R₂=Acm, R₃=H (FIG. 4B, 4); R₁=Acm, R₂═H, R₃=NBzl (FIG. 4B, 5); and R₁=Acm, R₂=NBzl, R₃=H (FIG. 4B, 6); a schematic representation of the syntheses of the six linaclotide analogues (FIG. 4C) via treatment with DSF for 10 s, then UV (350 nm) for 8 min and Pd^{II}/DTC+DSF for 5 min; HPLC comparison (FIG. 4D) of (1) R₁=H, R₂=Acm, R₃=NBzl. (2) R₁=Acm, R₂═H, R₃=NBzl (3) R₁=H, R₂=NBzl, R₃=Acm. (4) R₁=NBzl, R₂═H, R₃=Acm. (5) R₁=Acm, R₂=NBzl, R₃=H, (6) R₁=NBzl, R₂=Acm, R₃=H. c) R₁=NBzl, R₂═H, R₃=Acm. d) R₁=NBzl, R₂=Acm, R₃=H. e) R₁=Acm, R₂=H, R₃=NBzl. f) R₁=Acm, R₂=NBzl, R₃=H, the main peak corresponds to fully oxidized linaclotide with the observed mass: 1525.1±0.1 Da, calcd: 1525.1 Da (average isotopes); and HPLC and mass analyses (FIG. 4E) of the purified peptides for the six analogues of linaclotide: the main peak corresponds to the desired peptides, with the observed mass 3297.6±0.2 Da, calcd 3297.4 Da (average isotopes). (1) R₁=H, R₂=Acm, R₃=NBzl. (2) R₁=Acm, R₂═H, R₃=NBzl (3) R₁=H, R₂=NBzl, R₃=Acm. (4) R₁=NBzl, R₂═H, R₃=Acm. (5) R₁=Acm, R₂=NBzl, R₃=H. (6) R₁=NBzl, R₂=Acm, R₃=H;

FIG. 5 presents HPLC and mass analyses of the purified peptides for the six analogues of EETI-II: the main peak corresponds to the desired peptides, with the observed mass 3297.6±0.2 Da, calcd: 3297.4 Da (average isotopes); R₁=H, R₂=Acm, R₃=NBzl (1), R₁=Acm, R₂═H, R₃=NBzl (2), R₁=NBzl, R₂=Acm, R₃=H (3), R₁=NBzl, R₂═H, R₃=Acm (4), R₁=H, R₂=NBzl, R₃=Acm (5), and R₁=Acm, R₂=NBzl, R₃=H (6);

FIGS. 6A-E present a scheme of Conotoxin mr3e model peptide synthesis (FIG. 6A) and representative HPLC-MS analysis of Conotoxin mr3e model peptide at: reaction at time zero (FIG. 6B), the main peak corresponds to reduced mr3e with the observed mass 2161.6±0.1 Da, calcd: 2163.1 Da (average isotopes); reaction after 10 s (FIG. 6C), the main peak corresponds to mr3e with single disulfide bond with the observed mass 2159.6±0.1 Da, calcd: 2161.1 Da (average isotopes); reaction after 8 min (FIG. 6D): the main peak corresponds to mr3e with two disulfide bonds with the observed mass 1887.5±0.2 Da, calcd: 1889.1 Da (average isotopes); and reaction after 13 min (FIG. 6E): the main peak corresponds to mr3e with three disulfide bonds with the observed mass 1743.6±0.1 Da, calcd: 1745.1 Da (average isotopes). R₁=NBzl, R₂=Acm;

FIGS. 7A-E present a scheme of plectasinMet13Ala synthesis (FIG. 7A) and HPLC-MS analysis of: reaction at time zero (FIG. 7B), the main peak corresponds to reduced plectasin with the observed mass 4758.9±0.1 Da, calcd: 4758.9 Da (average isotopes); reaction after 10 s (FIG. 7C), the main peak corresponds to plectasin with single disulfide bond with the observed mass 4756.4±0.1 Da, calcd: 4756.9 Da (average isotopes); reaction after 8 min (FIG. 7D): the main peak corresponds to plectasin with two disulfide bonds with the observed mass 4484.5±0.2 Da, calcd: 4484.9 Da (average isotopes); and reaction after 13 min (FIG. 7E): the main peak corresponds to plectasin with three disulfide bonds with the observed mass 4340.6±0.1 Da, calcd: 4340.9 Da (average isotopes). R₁=NBzl, R₂=Acm;

FIGS. 8A-B present bar graphs of: antimicrobial activity assay for plectasin WT (1) and plectasin Met13Ala (2) (FIG. 8A): absorbance monitoring at 600 nm for MRSA growth after 17 h. LB was used as medium in the assay. Data are represented as mean±SD, n=3 biologically independent samples, error bars represent the SD. MIC=5 μM; and MIC comparison for the tested antimicrobial agents (FIG. 8B): plectasinMet13Nle (MIC=10 μM), plectasin WT (MIC=5 μM), plectasinMet13Nva (MIC=2.5 μM), plectasinMet13Abu (MIC=2.5 μM), TMP (MIC=2 μM) and plectasinMet13Ala (MIC=1.25 μM);

FIGS. 9A-C present HPLC and mass analyses of EETI-II disulfide bonds formation via folding process (unprotected and reduced EETI-II peptide dissolved in sodium phosphate buffer (0.2 M, pH 8) and oxidized by stirring open to the atmosphere): time zero (FIG. 9A): the main peak corresponds to the reduced EETI-II with the observed mass: 2884.5±0.3 Da, calcd: 2885.4 Da (average isotopes); Time=24 h (FIG. 9B): peak (a) corresponds to EETI-II bearing two disulfide bonds with the observed mass: 2880.5±0.2 Da, calcd: 2881.4 Da (average isotopes), and peak (b) corresponds to the fully oxidized EETI-II with the observed mass: 2878.5±0.2 Da, calcd: 2879.4 Da (average isotopes); Time=72 h (FIG. 9C): peak (a) corresponds to EETI-II bearing two disulfide bonds with the observed mass: 2880.5±0.2 Da, calcd: 2881.4 Da (average isotopes), and peak (b) corresponds to the fully oxidized EETI-II with the observed mass: 2878.5±0.2 Da, calcd: 2879.4 Da (average isotopes);

FIGS. 10A-B present HPLC and mass analyses of crude (FIG. 10A): the main peak corresponds to the desired peptide; and purified (FIG. 10B): the main peak corresponds to the desired peptide, with the observed mass 7991.3±0.1 Da, calcd: 7992.0 Da (average isotopes); and

FIGS. 11A-G present a scheme of RANTES synthesis (FIG. 11A), HPLC-ESI MS analyses of: reaction at time zero (FIG. 11B), the main peak corresponds to reduced RANTES modified with two Acm groups and two free Cys with the observed mass 7991.3±0.1 Da, calcd: 7992.0 Da (average isotopes); Reaction after 1 min (FIG. 11C): the main peak corresponds to RANTES bearing one disulfide bond and modified with two Acm with the observed mass 7989.6±0.3 Da, calcd: 7990.0 Da (average isotopes); reaction after 5 min (FIG. 11D):HPLC-ESI MS, the main peak corresponds to RANTES bearing two disulfide bonds with the observed mass 7844.6±0.1 Da, calcd: 7846.0 Da (average isotopes); purification and folding (FIG. 11E): the main peak corresponds to RANTES bearing two disulfide bonds with the observed mass 7844.7±0.1 Da, calcd: 7846.0 Da (average isotopes); commercially available native RANTES (FIG. 11F): analyses with the observed mass 7843.9±0.1 Da, calcd: 7846.0 Da (average isotopes), and CD spectrum (FIG. 11G) of the synthetic RANTES.

### DETAILED DESCRIPTION OF THE INVENTION

According to one aspect of the invention, provided herein a method for forming a disulfide bond, comprising: (a) providing a peptide comprising a first cysteine pair, wherein each thiol of the first cysteine pair is protected by a photocleavable protecting group; (b) exposing the peptide to light for a time period sufficient for de-protection of the photocleavable protecting group; (c) adding a sufficient amount of a reagent comprising disulfiram (DSF), diethyldithiocarbamate (DTC) or both, thereby forming a first disulfide bond; wherein the step b and the step c are performed simultaneously or subsequently. In some embodiments, the terms “disulfide bond”, “first disulfide bond”, and “second disulfide bond”, including any grammatical form thereof, refers to one or more intramolecular disulfide bond. In some embodiments, the term “intramolecular disulfide bond” is well-known in the art, and refers to a disulfide bond formed between a pair of thiols present within the same molecule.

### Photocleavable PG

In some embodiments, there is a method for forming a disulfide bond, comprising: (a) providing a peptide comprising at least two cysteine amino acids, wherein each thiol of the at least two cysteine amino acids is protected by a photocleavable protecting group; (b) exposing the peptide to light for a time period sufficient for de-protection of the photocleavable protecting group; (c) adding a sufficient amount of a reagent capable of promoting disulfide bond formation wherein the step b and the step c are performed simultaneously or subsequently. In some embodiments, the step b and the step c (e.g. the steps involving a chemical reaction) of any one of the methods disclosed herein are performed in one pot. In some embodiments, the method of the invention is for forming one or more (e.g. 2, 3, 4, 5, 6, etc.) intramolecular disulfide bond(s).

In some embodiments, there is a method for forming an intramolecular disulfide bond comprising: (a) providing a molecule comprising at least two thiol residues, wherein each thiol residue is protected by a photocleavable protecting group; and performing the steps b to c, as described hereinabove, thereby forming the intramolecular disulfide bond. In some embodiments, the molecule is a peptide or a peptoid. In some embodiments, the molecule is a small molecule comprising a plurality of thiols (e.g. alkylthiol residues). In some embodiments, the molecule is a peptide or a peptoid comprising a pair of cysteine amino acids, wherein each thiol of the at least two cysteine amino acids is protected by the photocleavable protecting group.

In some embodiments, there is a method for forming an intramolecular disulfide bond comprising: (a) providing a molecule comprising at least two thiol residues, wherein each thiol residue is protected by a photocleavable protecting group; and performing the steps b to c, as described hereinabove, thereby forming the intramolecular disulfide bond. In some embodiments, the molecule is a peptide or a peptoid. In some embodiments, the molecule is a small molecule comprising a plurality of thiols (e.g. alkylthiol residues).

In another aspect, provided herein a molecule comprising at least two pairs of thiol residues, wherein the first pair is protected by a photocleavable PG and the second pair is protected by a photostable PG. In some embodiments, the photocleavable PG and the photostable PG are orthogonal. In some embodiments, the molecule further comprises a deprotected thiol residue. In some embodiments, the molecule further comprises a deprotected pair of thiol residues.

In some embodiments, the photostable PG and the photocleavable PG are as described herein. In some embodiments, the molecule is a small molecule. In some embodiments, the molecule is a peptide or peptoid. In some embodiments, the molecule is a peptide comprising a first pair of cysteines protected by a photocleavable PG and a second pair of cysteines protected by a photostable PG, wherein the photostable PG and the photocleavable PG are as described herein. In some embodiments, the molecule is a peptide comprising a first pair of deprotected cysteines and a second pair of cysteines protected by a photostable PG or by a photocleavable PG. In some embodiments, the molecule is a peptide comprising a first pair of cysteines protected by a photocleavable PG, a second pair of cysteines protected by a photostable PG and an additional pair of deprotected cysteines.

In some embodiments, the reagent capable of promoting disulfide bond formation comprises a disulfanyl (also used herein as a disulfide). In some embodiments, the reagent capable of promoting disulfide bond formation catalyzes disulfide bond formation. In some embodiments, the reagent capable of promoting disulfide bond formation undergoes a redox reaction with one or more thiols. In some embodiments, the reagent oxidizes one or more thiol(s), so as to result in a disulfide bond.

In some embodiments, the reagent capable of promoting disulfide bond formation comprises a disulfide, a diselenide, a thiol, a sulfide, a thiocarbonate, a dithiocarbonate, thiocarbamate, or a dithiocarbamate, including any combination, a disulfanyl derivate, a metal complex or a salt thereof. In some embodiments, the term “reagent capable of promoting disulfide bond formation” and the term “disulfide catalyst” are used herein interchangeably.

In some embodiments, the reagent capable of promoting disulfide bond formation comprises a dithiocarbamate, or any disulfanyl derivate thereof (e.g. disulfiram) or any combination thereof. In some embodiments, the reagent capable of promoting disulfide bond formation comprises disulfiram (DSF).

In some embodiments, peptide comprising two cysteine amino acids protected by the same photocleavable protecting group. In some embodiments, peptide comprising two cysteine amino acids protected by different photocleavable protecting groups.

In some embodiments, the peptide further comprises an additional cysteine, wherein a thiol group of the additional cysteine is protected by an orthogonal protecting group (PG). In some embodiments, the peptide further comprises a plurality of additional cysteines protected by an orthogonal PG. In some embodiments, each thiol group of the one or more additional cysteine is protected by an orthogonal PG.

As used herein the term “orthogonal” refers to ability of a PG to remain intact under deprotecting conditions of another PG. Such orthogonal PG pairs are well known in the art, such as Boc and Fmoc.

In some embodiments, the orthogonal PG is stable under conditions sufficient for deprotection of the photocleavable protecting group. In some embodiments, the orthogonal PG is stable under exposure to light having a wavelength suitable for deprotection of the photocleavable protecting group. In some embodiments, the orthogonal PG remains stable under condition of the method disclosed herein. In some embodiments, at least 90%, at least 95%, at least 97%, at least 99%, at least 99.9% of the orthogonal PG remains intact under condition of the method disclosed herein. In some embodiments, the orthogonal PG is a photostable PG.

In some embodiments, the light has an appropriate wavelength suitable for deprotecting the photocleavable PG. In some embodiments, the light has a wavelength being within the absorption range of the photocleavable PG. In some embodiments, the light is a UV-light.

In some embodiments, the light is at a wavelength (also referred to herein as the appropriate wavelength) between 200 and 600 nm, between 300 and 370 nm, between 340 and 360 nm, including any range between.

In some embodiments, the photocleavable PG comprises a nitro-benzyl group. In some embodiments, the photocleavable PG is o-nitrobenzyl (NBzl).

In some embodiments, the method is performed at a pH value ranging between 1 and 14, between 1 and 5, between 5 and 8, between 6.5 and 7.5, between 6 and 8, between 6.8 and 7.2, between 8 and 10, between 10 and 14, including any range between. In some embodiments, the step a comprises providing a solution comprising the peptide. In some embodiments, the step a further comprises adjusting pH of the solution, so as to obtain a solution having a pH value being between 5 and 8. In some embodiments, the solution is an aqueous solution. In some embodiments, the solution is an aqueous buffer. In some embodiments, the steps a to c are performed at pH as described herein (e.g. between 5 and 8). In some embodiments, the method is performed at pH of between 5 and 8, between 6.5 and 7.5, between 6 and 8, between 6.8 and 7.2 including any range between.

In some embodiments, step b of the method comprises exposing the peptide to light for a time period (also used herein as the irradiation time) and or intensity sufficient for deprotection of the photocleavable protecting group. In some embodiments, the appropriate wavelength, the sufficient intensity and the sufficient irradiation time can be estimated based on standard experimentation procedures which are well known in the art. For example, the deprotection efficiency can by estimated by calculating the concentration (e.g. via UV/Vis absorption) of the deprotected PG in the deprotection solution. In some embodiments, as used herein the term “deprotection” comprises at least 70%, at least 80%, at least 90%, at least 95%, at least 99% deprotection of the PG.

In some embodiments, the time period (e.g. irradiation time) sufficient for deprotection is between 0.5 and 20 min, between 2 and 10 min, between 5 and 10 min, including any range between.

In some embodiments, step b of the method comprises exposing the peptide to light for a time period (e.g. irradiation time) greater than the time period sufficient for deprotection of the photocleavable PG.

In some embodiments, the step b and the step c (e.g. the steps involving a chemical reaction) of any one of the hereindisclosed methods are performed simultaneously or subsequently.

In some embodiments, step b and step c of the method are performed simultaneously. In some embodiments, the method disclosed herein comprises: (a) providing a peptide comprising at least two cysteine amino acids, wherein each thiol of the at least two cysteine amino acids is protected by a photocleavable PG; (b) adding a sufficient amount of a reagent capable of promoting disulfide bond formation; and (c) exposing the peptide to light for a time period sufficient for de-protection of the photocleavable PG.

In some embodiments, the sufficient amount of a reagent capable of promoting disulfide bond formation comprises between 2 and 20, between 2 and 5, between 5 and 10, between 10 and 15, between 15 and 20 molar equivalents (relative to the molecule and/or the peptide).

In some embodiments, the disulfide bond formed by the method of the invention remains stable. In some embodiments, the disulfide bond formed by the method of the invention is substantially devoid of reshuffling (e.g. an inter- or intramolecular reaction between disulfide bonds).

In some embodiments, the method of the invention is performed in one-pot. The term “simultaneously” is further meant to refer to fact that the products of the corresponding step are being produced contemporaneously, i.e. happening in, or are associated with, the same period of time. For example, when the abovementioned steps (a) to (c) are performed simultaneously the deprotection of the PG and the formation of the disulfide bond occur contemporaneously.

In some embodiments, the term “one pot” is meant to refer to a synthesis (or to a process) being carried out in a single reaction vessel, typically, but not exclusively, without removing therefrom any intermediate products (e.g. without purification of an intermediate).

In another aspect, there is provided a compound of interest (e.g. a peptide) synthesized according to the method disclosed herein. In some embodiments, the compound of interest (e.g. a peptide) synthesized according to the method disclosed herein, comprises trace amounts of: (i) reagent comprising a disulfide, a diselenide, a thiol, a sulfide, a thiocarbonate, a dithiocarbonate, thiocarbamate, or a dithiocarbamate, including any combination, a disulfanyl derivate, including any metal complex or a salt thereof, and/or of (ii) the disulfide catalyst. In some embodiments, the reagent is or comprises glutathione, disulfiram (DSF), RRN—C(═S)—S—S—C(═S)—NRR, RRN—C(═S)—SH or a salt thereof, and diethyldithiocarbamate (DTC), including any combination thereof, wherein each R is as described herein.

In some embodiments, there is provided a composition comprising the compound of interest synthesized according to the method disclosed herein. In some embodiments, the composition is a pharmaceutical composition. In some embodiments, the pharmaceutical composition comprises a pharmaceutically effective amount of the compound of interest, and optionally comprises a pharmaceutically acceptable carrier.

In some embodiments, the term “trace amounts” refers to residual amounts of the reagent present within the composition disclosed herein. In some embodiments, residual amounts refer to a weight content of the reagent within the composition of at most 0.5%, at most 0.1%, at most 0.05%, at most 0.01%, at most 0.005%, at most 0.001%, including any range between. In some embodiments, the term “trace amounts” refers to any regulatory acceptable trace level (or w/w concentration) of the reagent within the composition or within the pharmaceutical composition. In some embodiments, the composition and/or the peptide of interest is a pharmaceutical grade composition and/or is a pharmaceutical grade peptide, respectively. In some embodiments, the composition and/or the peptide of interest is pharmaceutically pure. In some embodiments, the compound of interest (e.g. the peptide) comprises at least 90%, at least 93%, at least 95%, at least 97%, at least 99%, or 100% structural homology to a known crystal structure of the same peptide.

In some embodiments, the compound of interest is a peptide, wherein the peptide comprises at least 90%, at least 93%, at least 95%, at least 97%, at least 99%, or 100% structural homology to a known crystal structure of the same peptide (e.g. a peptide having the same sequence, also referred to herein as the “native peptide”), and/or retains at least 90%, at least 93%, at least 95%, at least 97%, at least 99%, of the biological activity of the native peptide. A skilled artisan will appreciate, that a correct folding of a peptide (e.g. due to the formation of specific intramolecular disulfide bonds) substantially predetermines biological activity of the peptide. Accordingly, it is postulated that a peptide having disulfide bonds formed between the corresponding cysteine pairs (and identical to the disulfide bonds formed within the native peptide) will substantially retain the biological activity of the native peptide.

In some embodiments, there is provided a kit comprising at least one disulfide catalyst. In some embodiments, the kit comprises a first composition comprising the disulfide catalyst. In some embodiments, a concentration of the disulfide catalyst within the first composition is at least 0.1 uM, at least 1 uM, at least 10 uM, at least 100 uM, at least 0.1 mM, at least 10 mM, at least 100 mM, at least 0.1M, including any range between.

In some embodiments, the kit comprises a second composition comprising a precursor (e.g. a peptide) having at least 2, at least 4, at least 6 thiols (protected or deprotected). In some embodiments, the precursor comprises 1, 2, 3, 4, 5, 6, or more thiol pairs (e.g. cysteines) optionally protected by the photocleavable PG. In some embodiments, the first composition and/or the second composition comprises a buffer.

As used herein, the term “compound of interest” refers to a molecule comprising one or more intramolecular disulfide bonds. In some embodiments, the compound of interest is or comprises a peptide. In some embodiments, the compound of interest is or comprises a molecule having a biological activity. In some embodiments, the compound of interest comprises a peptide having a plurality of intramolecular disulfide bonds. In some embodiments, the compound of interest comprises a peptide having at least 2 intramolecular disulfide bonds. In some embodiments, the compound of interest comprises a peptide having at least 3 intramolecular disulfide bonds. In some embodiments, the compound of interest comprises a peptide having at least 4 intramolecular disulfide bonds.

In some embodiments, a concentration of the disulfide catalyst within the first composition is between 2 and 20, between 2 and 5, between 5 and 10, between 10 and 15, between 15 and 20 molar equivalents relative to one or more thiol pair within the precursor (e.g. peptide).

In some embodiments, the kit comprises the first composition and optionally comprises at least one of: (i) a precursor (e.g. a peptide) having at least 1, at least 2, at least 3, at least 4, at least 6 thiol pairs (protected or deprotected) including any range between; and (ii) a cleavage agent (e.g. an acid solution, such as TFA solution).

### Photostable PG

In some embodiments, there is a method for forming an intramolecular disulfide bond comprising: (a) providing a molecule (also used herein as “compound of interest”) comprising at least two thiol residues, wherein each thiol residue is protected by a photostable PG; and (b) adding a sufficient amount of the reagent capable of promoting disulfide bond formation to the molecule, and providing the molecule under conditions sufficient for deprotecting the photostable PG, thereby forming intramolecular disulfide bond between thiol residues of the molecule. In some embodiments, the molecule is a peptide or a peptoid. In some embodiments, the molecule is a small molecule comprising a plurality of thiols (e.g. alkylthiol residues). In some embodiments, the molecule is a peptide or a peptoid comprising at least one pair of cysteine amino acids, wherein each thiol of the at least two cysteine amino acids is protected by photostable PG.

In some embodiments, the reagent capable of promoting disulfide bond formation is as described herein. In some embodiments, the reagent capable of promoting disulfide bond formation comprises DSF.

In some embodiments, the photostable PG is as described hereinbelow. In some embodiments, the photostable PG is acetamidomethyl (Acm).

In some embodiments, the conditions sufficient for deprotecting the photostable PG are as described herein. In some embodiments, the conditions sufficient for deprotecting the photostable PG comprise adding a transition metal catalyst to the molecule (e.g. reaction mixture), as described herein.

In some embodiments, the transition metal catalyst comprises a transitions metal (e.g. in an elemental state or in an oxidized state) and/or a salt thereof. In some embodiments, the transition metal is selected from the group consisting of Pt, Pd, Ru, Cu, Ni, Co, Ti, Zn and Ag or any combination thereof. In some embodiments, the transition metal catalyst comprises Pd(II) salt, such as palladium halide salt. In some embodiments, the transition metal catalyst comprises a salt of a transition metal, wherein the salt comprises a transition metal cation and an anion. Various anions suitable for salt formation with a transition metal cation are well-known in the art (e.g. halide, acetate, nitrate, etc.).

In some embodiments, the conditions sufficient for deprotecting the photostable PG further comprise maintaining pH of the reaction mixture, as described hereinbelow.

In some embodiments, the compound of interest (e.g. a peptide) synthesized according to the method disclosed herein, comprises trace amounts of: (i) a thiocarbamic acid and/or a salt thereof (e.g. diethyldithiocarbamic acid or a salt thereof), and/or of (ii) the disulfide catalyst, and/or of (iii) the transition metal catalyst, including any salt or any derivative thereof (e.g. transition metal catalyst in a reduced state or in an elemental state, a salt thereof, and/or a hydroxide thereof).

In some embodiments, there is provided a kit comprising at least one disulfide catalyst and at least one transition metal catalyst. In some embodiments, the kit comprises a first composition comprising the disulfide catalyst, and a second composition comprising the transition metal catalyst. In some embodiments, a concentration of the disulfide catalyst and/or of the transition metal catalyst within the first composition and within the second composition, respectively, is at least 0.1 uM, at least 1 uM, at least 10 uM, at least 100 uM, at least 0.1 mM, at least 10 mM, at least 100 mM, at least 0.1M, including any range between.

In some embodiments, the kit comprises a third composition comprising a precursor (e.g. a peptide) having at least 2, at least 4, at least 6 thiols (wherein at least 2 thiols are protected by a PG). In some embodiments, the precursor comprises 2, 3, 4, 5, 6, or more of thiol pairs (e.g. cysteines) optionally protected by the photostable PG. In some embodiments, the first composition, the second composition and/or the third composition comprises a buffer.

In some embodiments, a concentration of the disulfide catalyst within the first composition is between 2 and 20, between 2 and 5, between 5 and 10, between 10 and 15, between 15 and 20 molar equivalents relative to one or more thiol pairs within the precursor (e.g. peptide). In some embodiments, a concentration of the transition metal catalyst within the second composition is between 2 and 20, between 2 and 5, between 5 and 10, between 10 and 15, between 15 and 20 molar equivalents relative one or more thiol pairs within the precursor (e.g. peptide).

In some embodiments, the kit further comprises instructions for mixing and optionally diluting the first composition and/or the second composition with a peptide of interest. In some embodiments, the kit comprises instructions of mixing a predetermined amount of the first composition and/or the second composition with a compound (e.g. peptide) of interest, so as to result in an efficient formation of one or more intramolecular disulfide bonds.

In some embodiments, the predetermined amount comprise an amount of the disulfide catalyst and/or of the transition metal catalyst between 2 and 20, between 2 and 5, between 5 and 10, between 10 and 15, between 15 and 20 molar equivalents relative to at least one thiol (e.g. cysteine) pair within the compound of interest.

In some embodiments, the kit comprises the first composition and the second composition and optionally comprises at least one of: (i) a precursor (e.g. a peptide) having at least 1, at least 2, at least 3, at least 4, at least 5, at least 6 thiol pairs (protected or deprotected); and (ii) a cleavage agent (e.g. an acid solution, such as TFA solution).

In some embodiments, any of the kits disclosed herein are for the formation of one or more intramolecular disulfide bond(s) within a compound of interest.

In some embodiments, the method of the invention is described in greater detail in the Appendix incorporated herein.

### Multi-Step Process

In another aspect, there is a method of forming a plurality of intramolecular disulfide bonds, comprising (a) providing a molecule comprising (i) a pair of deprotected thiol residues, and (ii) a first pair of thiol residues protected by a first protecting group and optionally (iii) a second pair of thiol residues protected by a second protecting group; (b) adding to the molecule a sufficient amount of the reagent capable of promoting disulfide bond formation, thereby forming a first intramolecular disulfide bond between the deprotected thiol residues; (c) adding a sufficient amount of the reagent capable of promoting disulfide bond formation to the molecule, and providing the molecule under conditions sufficient for deprotecting the first protecting group, thereby forming a second intramolecular disulfide bond between first pair of thiol residues; wherein: the first protecting group and the second protecting group are orthogonal. In some embodiments, the steps b and c are performed in one pot. In some embodiments, the reagent capable of promoting disulfide bond formation is as described herein. In some embodiments, the method further comprises step d, comprising adding a sufficient amount of the reagent capable of promoting disulfide bond formation to the molecule and providing the molecule under conditions sufficient for deprotecting the second protecting group, thereby inducing formation of a second disulfide bond between the second pair of thiol residues. In some embodiments, the step d is performed prior to the step c or subsequent to the step c. In some embodiments, the steps a to d are performed in one pot. In some embodiments, the molecule is a peptide or a peptoid. In some embodiments, the molecule is a small molecule comprising a plurality of thiols (e.g. alkylthiol residues).

In some embodiments, at least two of the steps a to d are performed in one pot. In some embodiments, at least three of the steps a to d are performed in one pot. In some embodiments, at least two of the steps b to d are performed in one pot. In some embodiments, the steps b to d are performed in one pot.

In some embodiments, the method for forming a plurality of disulfide bonds within a peptide, comprising: (a) providing a peptide comprising (i) a deprotected cysteine pair, (ii) a first cysteine pair protected by a first protecting group and optionally (iii) a second cysteine pair protected by a second protecting group; (b) adding to the peptide a sufficient amount of a reagent comprising a disulfide, a diselenide, a thiol, a sulfide, a thiocarbonate, a dithiocarbonate, thiocarbamate, or a dithiocarbamate, including any combination, a disulfanyl derivate, a metal complex or a salt thereof; thereby forming a first disulfide bond between thiols of the deprotected cysteine pair; (c) adding a sufficient amount of the reagent to the peptide and providing the peptide under conditions sufficient for deprotecting the first protecting group, thereby forming a second disulfide bond between thiols of the first cysteine pair; wherein: the first protecting group and the second protecting group are orthogonal; and the steps b and c are performed in one pot.

In some embodiments, there is a method for forming at least two disulfide bonds. In some embodiments, the method comprises step (a) of providing a peptide comprising (i) a deprotected cysteine pair, (ii) a first cysteine pair protected by a first protecting group; step (b) of adding to the peptide a sufficient amount of the reagent capable of promoting disulfide bond formation, thereby forming a first disulfide bond between thiols of the deprotected cysteine pair; (c) adding a sufficient amount of reagent capable of promoting disulfide bond formation to the peptide and providing the peptide under conditions sufficient for deprotecting the first protecting group, thereby forming a second disulfide bond between thiols of the first cysteine pair. In some embodiments, the steps b and c are performed in one pot. In some embodiments, the steps b and c are performed sequentially or subsequently. In some embodiments, the reagent capable of promoting disulfide bond formation is as described hereinabove. In some embodiments, the reagent capable of promoting disulfide bond formation is a disulfide. In some embodiments, the reagent capable of promoting disulfide bond formation is DSF.

In some embodiments, the step of forming the first disulfide bond comprises performing the steps a and b at pH of between 1 and 14, between 1 and 5, between 5 and 8, between 6.5 and 7.5, between 6 and 8, between 6.8 and 7.2, between 8 and 10, between 10 and 14, including any range between.

In some embodiments, the method for forming the first disulfide bond comprises performing the steps a and b under appropriate conditions. In some embodiments, the appropriate conditions comprise a time period between 0.1 and 20 min, between 1 second (s) and 20 min, between 1 s and 1 min, between 1 and 5 min, between 5 and 10 min, between 10 and 15 min, between 15 and 20 min, between 5 and 20 min, including any range between, or more. In some embodiments, the appropriate conditions comprise a time period of less than 20 min, less than 15 min, less than 10 min, less than 5 min, less than 3 min, less than 2 min, less than 1 min, including any range between.

In some embodiments, the appropriate conditions comprise a temperature of the reaction mixture being between 10 and 50° C., between 25 and 40° C., between 10 and 40° C., between 10 and 25° C., between 10 and 30° C., between 20 and 40° C., between 20 and 50° C., between 10 and 50° C., between 30 and 40° C., including any range between.

In some embodiments, the sufficient amount of the reagent capable of promoting disulfide bond formation is as described hereinabove. In some embodiments, the sufficient amount of the reagent capable of promoting disulfide bond formation comprises a molar ratio between the reagent and the peptide being between 5 and 20, between 5 and 10, between 10 and 15, between 15 and 20, including any range between.

In some embodiments, the first disulfide bond is stable under conditions of the method (e.g. step c and/or step d), wherein stable is as described hereinabove.

### Method 1 (Photocleavable PG)

In some embodiments, the peptide comprises (i) a deprotected cysteine pair, and (ii) a first cysteine pair protected by a photocleavable PG. In some embodiments, the step of forming the first disulfide bond between thiols of the deprotected cysteine pair (also referred to as a first intramolecular disulfide bond) is as described hereinabove. In some embodiments, the peptide further comprises an additional cysteine, wherein a thiol group of the additional cysteine is protected by an orthogonal protecting group (PG). In some embodiments, the peptide further comprises a plurality of additional cysteines protected by an orthogonal PG. In some embodiments, each thiol group of the, one or more additional cysteine is protected by an orthogonal PG. In some embodiments, the peptide further comprises at least one amino acid protected by an orthogonal PG. In some embodiments, the orthogonal PG is as described hereinabove.

In some embodiments, the step of forming a second disulfide bond between thiols of the first cysteine pair (also referred to as a second intramolecular disulfide bond) comprises adding a sufficient amount of the reagent capable of promoting disulfide bond formation to the peptide; and providing the peptide under conditions sufficient for deprotecting the photocleavable PG.

In some embodiments, the light has a wavelength suitable for deprotecting the photocleavable PG. In some embodiments, the light has a wavelength being within the absorption range of the photocleavable PG. In some embodiments, the light is a UV-light.

In some embodiments, the light is at a wavelength between 200 and 600 nm, between 300 and 370 nm, between 340 and 360 nm, including any range between.

In some embodiments, the photocleavable PG comprises a nitro-benzyl group. In some embodiments, the photocleavable PG is o-nitrobenzyl (NBzl).

In some embodiments, the method is performed at a pH value ranging between 5 and 8, between 6.5 and 7.5, between 6 and 8, between 6.8 and 7.2 including any range between. In some embodiments, the method comprises providing a solution comprising the peptide. In some embodiments, the step a further comprises adjusting pH of the solution, so as to obtain a solution having a pH value being between 5 and 8. In some embodiments, the solution is an aqueous solution. In some embodiments, the solution is an aqueous buffer. In some embodiments, the method is performed at pH as described herein (e.g. between 5 and 8). In some embodiments, the method is performed at pH of between 5 and 8, between 6.5 and 7.5, between 6 and 8, between 6.8 and 7.2 including any range between.

In some embodiments, the method comprises exposing the peptide to light for a time period sufficient for deprotection of the photocleavable protecting group. In some embodiments, deprotection comprises at least 70%, at least 80%, at least 90%, at least 95%, at least 99% deprotection of the PG.

In some embodiments, the time period sufficient for deprotection is between 0.5 and 20 min, between 2 and 10 min, between 5 and 10 min, including any range between.

In some embodiments, the method comprises exposing the peptide to light for a time period greater than the time period sufficient for deprotection of the photocleavable PG, thereby deprotecting the photocleavable PG. In some embodiments, deprotecting the photocleavable PG and forming the second intramolecular bond is performed simultaneously.

In some embodiments, the steps involving a chemical reaction of any one of the hereindisclosed methods are performed simultaneously or subsequently.

In some embodiments, (i) the addition of the reagent capable of promoting disulfide bond formation and (ii) exposing the peptide to conditions sufficient for deprotection of the photocleavable PG are performed simultaneously.

In some embodiments, the sufficient amount of a reagent capable of promoting disulfide bond formation comprises between 2 and 20, between 2 and 5, between 5 and 10, between 10 and 15, between 15 and 20 molar equivalents (relative to at least one thiol pair of the compound of interest).

In some embodiments, the step of forming the first intramolecular bond and the step of forming the second intramolecular bond are performed subsequently. In some embodiments, the step of forming the first intramolecular bond and the step of forming the second intramolecular bond are performed in one pot.

### Method 2 (Photostable PG)

In some embodiments, the peptide comprises (i) a deprotected cysteine pair, and (ii) a first cysteine pair protected by a photostable PG. In some embodiments, the step of forming the first disulfide bond between thiols of the deprotected cysteine pair (also referred to as a first intramolecular disulfide bond) is as described hereinabove. In some embodiments, the peptide further comprises an additional cysteine, wherein a thiol group of the additional cysteine is protected by an orthogonal protecting group (PG). In some embodiments, the peptide further comprises a plurality of additional cysteines protected by an orthogonal PG. In some embodiments, each thiol group of the, one or more additional cysteine is protected by an orthogonal PG. In some embodiments, the peptide further comprises at least one amino acid protected by an orthogonal PG. In some embodiments, the orthogonal PG is as described hereinabove (e.g. stable under conditions for deprotecting the photostable PG).

In some embodiments, the step of forming a second disulfide bond between thiols of the first cysteine pair comprises adding a sufficient amount of the reagent capable of promoting disulfide bond formation to the peptide; and providing the peptide under conditions sufficient for deprotecting the photostable PG.

In some embodiments, the molecule is a small molecule comprising a plurality of thiols (e.g. alkylthiol residues). In some embodiments, the molecule is a peptide or a peptoid comprising a pair of cysteine amino acids, wherein each thiol of the at least two cysteine amino acids is protected by photostable PG.

In some embodiments, the reagent capable of promoting disulfide bond formation is as described herein. In some embodiments, the reagent capable of promoting disulfide bond formation comprises DSF.

In some embodiments, the photostable PG is as described hereinbelow. In some embodiments, the photostable PG is acetamidomethyl (Acm).

In some embodiments, the conditions sufficient for deprotecting the photostable PG comprise adding a transition metal catalyst to the molecule (e.g. reaction mixture), as described herein. In some embodiments, the conditions sufficient for deprotecting the photostable PG comprise adding a sufficient amount of the transition metal catalyst to the peptide and/or to a reaction mixture comprising same. In some embodiments, the sufficient amount of the transition metal catalyst comprises between 5 and 30 molar equivalents (relative to at least one thiol pair of the compound of interest). In some embodiments, the sufficient amount of the transition metal catalyst comprises between 10 and 20 molar equivalents.

In some embodiments, the transition metal catalyst comprises a transition metal, a complex or a salt thereof. Various transition metals are well known in the art (such as Ru, Pd, Ni, Pt, Rh, Co, Ti, Zn, Pt, Ag etc.).

In some embodiments, the transition metal catalyst is soluble within the reaction mixture. In some embodiments, the transition metal catalyst comprises Pd(II) salt. In some embodiments, the transition metal catalyst comprises Pd(II) halide.

In some embodiments, the conditions sufficient for deprotecting the photostable PG comprise maintaining pH of the reaction mixture prior to or subsequently to the transition metal catalyst addition. In some embodiments, the pH of the reaction mixture is maintained at a pH value below 2, below 1.5, below 1.3 below 1.2, below 1, including any range between.

In some embodiments, the conditions sufficient for deprotecting the photostable PG comprise a reaction time of between 1 and 10 min.

In some embodiments, the method comprises adding a complexing agent capable of forming a stable complex with the transition metal catalyst. In some embodiments, the complexing agent capable of inactivating the transition metal catalyst.

In some embodiments, the complexing agent comprises at least one of a thiol, a thiocarbonate, a dithiocarbonate, thiocarbamate, or a dithiocarbamate, including any disulfanyl derivate thereof. In some embodiments, the complexing agent comprises diethyldithiocarbamate (DTC). In some embodiments, an amount of the complexing agent is sufficient for substantially complexing or inactivating the transition metal catalyst. In some embodiments, the complexing agent comprises a thiol. In some embodiments, the complexing agent is or comprises glutathione.

In some embodiments, the complexing agent and the reagent disclosed herein, independently comprise one or more species. In some embodiments, the complexing agent and the reagent disclosed herein, are the same or different (species).

In some embodiments, the term thiol encompasses, HS—R or R—S—R, including any salt thereof, where each R is independently selected from hydrogen, alkyl, cycloalkyl, alkenyl, aryl, heteroaryl (bonded through a ring carbon) or heteroalicyclic (bonded through a ring carbon) or any combination thereof. In some embodiments, a thiol derivative encompasses any of disulfide, trisulfide, including any salt thereof, wherein disulfide and/or trisulfide is optionally substituted with one or two R, where each R is independently described herein.

In some embodiments, the term thiocarbonate encompasses ROC(═O)SH, ROC(═O)S⁻ anion, and/or CO₂S²⁻ anion, including any salt thereof, where each R is as defined above. In some embodiments, the term dithiocarbonate encompasses COS₂ anion, ROC(═S)SH, and/or ROC(═S)S⁻ anion, including any salt thereof, where each R is as defined above. In some embodiments, the term dithiocarbonate encompasses RSC(═O)—SR, and/or RSC(═O)S⁻ anion, including any salt thereof, where each R is as defined above. In some embodiments, a derivative of the thiocarbonate refers to a trithiocarbonate, encompassing RS—C(═S)—SR, and/or RSC(═S)S⁻ anion, where each R is as defined above; or to RS—S—C(═O)—OR, including any salt thereof, where each R is as defined above. In some embodiments, a sulfiram derivative of the thiocarbonate or of the dithiocarbonate refers to RO—C(═O)S—SC(═O)—OR, or to RS—C(═O)S—SC(═O)—SR respectively, where each R is as defined above.

In some embodiments, the term thiocarbamate encompasses R—O—C(═S)—NRR, or R—S—C(═O)—NRR, where each R is as defined above. In some embodiments, the term dithiocarbamate encompasses R—S—C(═S)—NRR, where each R is as defined above. In some embodiments, a sulfiram derivative of the dithiocarbamate refers to RS—C(═S)S—SC(═S)—SR, or RRN—C(═S)—S—S—C(═S)—NRR (e.g. disulfiram) where each R is as defined above.

In some embodiments, the term disulfiram derivative encompasses a dimer of the compound, wherein both portions of the dimer are bound via a disulfide bond. In some embodiments, the dimer is a symmetric molecule, with a symmetry axis located between two sulfur atoms of the disulfide bond. In some embodiments, the term disulfiram derivative encompasses a disulfide derivative of the compound, wherein the derivative is a symmetrical or an asymmetrical molecule.

In some embodiments, the complexing agent is added after deprotecting the photostable PG.

In some embodiments, the method further comprises maintaining pH of the reaction mixture, so as to provide the reaction mixture at pH of between 5 and 8, between 6 and 7.

In some embodiments, the method further comprises adding a sufficient amount of the reagent capable of promoting disulfide bond formation, thereby forming the second intramolecular bond. In some embodiments, the reagent capable of promoting disulfide bond formation is as described herein. In some embodiments, the sufficient amount of the reagent capable of promoting disulfide bond formation is as described hereinabove.

In some embodiments, the step of forming the first intramolecular bond and the step of forming the second intramolecular bond are performed subsequently. In some embodiments, the step of forming the first intramolecular bond and the step of forming the second intramolecular bond are performed in one pot.

In some embodiments, the first intramolecular bond and the second intramolecular bond are stable under conditions of the method described herein. In some embodiments, the photostable PG is stable under conditions for forming the first intramolecular bond.

In some embodiments, the peptide comprises (i) a first cysteine pair protected by a photocleavable PG, and (ii) a second cysteine pair protected by a photostable PG. In some embodiments, the method comprises (i) forming a first disulfide bond between thiols of the first cysteine pair, and subsequently (ii) forming a second disulfide bond between thiols of the second cysteine pair, wherein steps i and ii are performed in one pot. In some embodiments, the step i is as described herein (method I). In some embodiments, the step ii is as described herein (method II). In some embodiments, the steps I and ii are performed in a reversed order (e.g. step ii prior to step i).

### Method 3 (Formation of 3 S—S Bonds)

In some embodiments, there is a method for forming three or more disulfide bonds. In some embodiments, the method comprises step a of providing a peptide comprising (i) a deprotected cysteine pair, (ii) a first cysteine pair protected by a photocleavable PG and (iii) a second cysteine pair protected by a photostable PG; and adding to the peptide a sufficient amount of the reagent capable of promoting disulfide bond formation, thereby forming a first disulfide bond between thiols of the deprotected cysteine pair. In some embodiments, the step a is as described hereinabove (first intramolecular disulfide bond).

In some embodiments, the method comprises step b of adding a sufficient amount of reagent capable of promoting disulfide bond formation to the peptide and exposing the peptide to light for a time period sufficient for deprotection of the photocleavable PG, thereby deprotecting the photocleavable PG and forming a second disulfide bond between thiols of the first cysteine pair. In some embodiments, the step b is performed as described above (Method 1).

In some embodiments, the method comprises step c of providing the peptide under conditions sufficient for deprotecting the photostable PG, and adding a sufficient amount of the reagent capable of promoting disulfide bond formation to the peptide; thereby forming a third disulfide bond between thiols of the second cysteine pair.

In some embodiments, the step c is performed as described above (Method 2). In some embodiments, the method of the invention comprises performing step c prior to performing step b.

In some embodiments, the photostable PG and the photocleavable PG are orthogonal. In some embodiments, the photostable PG is stable under conditions of step b. In some embodiments, the photocleavable PG is stable under conditions of step c. In some embodiments, the photostable PG and the photocleavable PG are stable under conditions of step a.

In some embodiments, the steps b and c are performed subsequently. In some embodiments, the steps b and c are performed in one pot. In some embodiments, the steps a to c are performed in one pot.

In some embodiments, the peptide further comprises an additional cysteine, wherein a thiol group of the additional cysteine is protected by an orthogonal protecting group (PG). In some embodiments, the peptide further comprises a plurality of additional cysteines protected by an orthogonal PG. In some embodiments, each thiol group of the, one or more additional cysteine is protected by an orthogonal PG. In some embodiments, the peptide further comprises at least one amino acid protected by an orthogonal PG. In some embodiments, the orthogonal PG is as described hereinabove.

In some embodiments, the photocleavable protecting group comprises NBzl. In some embodiments, the photostable PG comprise ACM.

### General

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

### Definitions

As used herein, the term “alkyl” describes an aliphatic hydrocarbon including straight chain and branched chain groups. Preferably, the alkyl group has 20 or less main-chain carbons (e.g. between 1-5, 5-10, 10-15, 15-20, including any range between). The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### General Methods

SPPS was carried out manually in syringes, equipped with teflon filters, purchased from Torviq or by using an automated peptide synthesizer (CS336X, CSBIO). If not differently described, all reactions were carried out at room temperature. Analytical grade N,N-dimethylformamide (DMF) was purchased from Bio-Lab Ltd. Commercial reagents were used without further purification. Resins were purchased from Creosalus, protected amino acids were purchased from GL Biochem and activating reagents [(2-(1H-benzotriazol-1-yl)-1,1,3,3-tetramethyluronium hexafluorophosphate (HBTU), hydroxybenzotriazole (HOBt), [(6-chlorobenzotriazol-1-yl)oxy-(dimethylamino)methylidene]-dimethylazanium hexafluorophosphate (HCTU), 1-[Bis(dimethylamino)methylene]-1H-1,2,3-triazolo[4,5-b]pyridinium 3-oxide hexafluorophosphate (HATU)] were purchased from Luxembourg Bio Technologies. Sodium diethyldithiocarbamate trihydrate, CAS no: 20624-25-3 was purchased from Merck. Tetraethylthiuram disulfide (disulfiram), CAS no: 97-77-8 was purchased from Merck. L-Glutathione reduced (GSH), CAS no: 70-18-8 was purchased from Merck. The Rayonet photochemical chamber reactor model RPR-200 was used for UV irradiation. 16 Rayonet lamps (350 nm, 24 watts, ˜3×10¹⁶ sec/cm³ photons) 12-inches in length were purchased from Southern New England Ultraviolet Company. Analytical HPLC was performed on a Thermo instrument (Dionex Ultimate 3000) using analytical columns Xbridge (waters, BEH300 C4, 3.5 μm, 4.6×150 mm) and XSelect (waters, CSH C18, 3.5 μm, 4.6×150 mm) at a flow rate of 1.2 mL/min. Preparative HPLC was performed on a Waters instrument using XSelect C18 10 μm 19×250 mm and semi-preparative HPLC was performed on a Thermo Scientific instrument (Spectra System SCM1000) using Jupiter C4 10 μm, 300 Å, 250×10 mm column, at a flow rate of 15 and 4 mL/min respectively. All synthetic products were purified by HPLC and characterized by mass spectrometry using LCQ Fleet Ion Trap (Thermo Scientific). All calculated masses have been reported as an average isotope composition. Buffer A: 0.1% TFA in water; buffer B: 0.1% TFA in acetonitrile.

### List of the Protected Amino Acids Used in Peptides Synthesis

The following amino acids were used in the peptide synthesis: Fmoc-Gly-OH, Fmoc-Ala-OH, Fmoc-Val-OH, Fmoc-Leu-OH, Fmoc-Ile-OH, Fmoc-Phe-OH, Fmoc-Pro-OH, Fmoc-His(Trt)-OH, Fmoc-Asn(Trt)-OH, Fmoc-Gln(Trt)-OH, Fmoc-Arg(Pbf)-OH, Fmoc-Lys(Boc)-OH, Fmoc-Tyr(tBu)-OH, Fmoc-Ser(tBu)-OH, Fmoc-Thr(tBu)-OH, Fmoc-Asp(OtBu)-OH, Fmoc-Glu(OtBu)-OH, Fmoc-Trp(Boc)-OH, Fmoc-Met-OH, Fmoc-Nle-OH, Fmoc-Nva-OH, Fmoc-Abu-OH, Fmoc-Cys(Acm)-OH, Fmoc-Cys(NBzl)-OH, Fmoc-Cys(Trt)-OH, Boc-Cys(Trt)-OH, Boc-Ser-OH, Boc-Gly-OH, Boc-Ile-OH, Fmoc-Asp(Boc)-Thr(ψMe,MePro)-OH, Fmoc-Asp(Boc)-Ser(ψMe,MePro)-OH, Fmoc-Tyr(Trt)-Thr(ψMe,MePro)-OH, Fmoc-Val-Thr(ψMe,MePro)-OH.

Table 1 presents the sequences of the prepared peptides and miniproteins.

TABLE 1
Sequences of the prepared
peptides and miniproteins

| | Peptide/ | |
|---|---|---|
| Entry | protein | Sequence |
| 1 | α-conotoxin- | ICCNPACGPKYSC |
| | SI | |
| 2 | EETI-II | GCPRILNleRCKQDSDCLAGCVCGPNGFCG |
| 3 | Plectasin | GFGCNGPWDEDDNleQCHNHCKS |
| | | IKGYKGGYCAKGGFVCKCY |
| | | GFGCNGPWDEDDMQCHNHCKS |
| | | IKGYKGGYCAKGGFVCKCY |
| | | GFGCNGPWDEDDAQCHNHCKS |
| | | IKGYKGGYCAKGGFVCKCY |
| | | GFGCNGPWDEDDAbuQCHNHCKS |
| | | IKGYKGGYCAKGGFVCKCY |
| | | GFGCNGPWDEDDNvaQCHNHCKS |
| | | IKGYKGGYCAKGGFVCKCY |
| 4 | Conotoxin- | VCCPFGGCHELCYCCD |
| | mr3e | |
| 5 | Linaclotide | CCEYCCNPACTGCY |
| 6 | RANTES | SPYSSDTTPCCFAYIARPLPRAHIKEYFYT |
| | | SGKCSNPAVVFVTRKNRQVCANPEKKWVRE |
| | | YINSLEMS |

### Synthesis of α-Conotoxin-SI Linear Peptide Bearing Cys(Acm) and Cys(NBzl)

The synthesis was carried out using Fmoc-SPPS on a Rink amide resin (0.26 mmol/g, 0.2 mmol scale). Peptide synthesis was performed on peptide synthesizer in presence of 4 equiv. of amino acid, HCTU and 8 equiv. of N,N′-diisopropylethylamine (DIEA). The pre-swollen resin was treated with 20% piperidine in DMF containing 0.1 mmol HOBt (3-5-3 min) to remove the Fmoc-protecting group. Deprotection and cleavage from the resin: The resin was washed with DMF, MeOH, DCM and dried. The peptide was cleaved using trifluoroacetic acid (TFA):triisopropylsilane (TIS):water (95:2.5:2.5) cocktail for 2 h. The cleavage mixture was filtered, and the combined filtrate was added dropwise to a 10-fold volume of cold ether and centrifuged. Fmoc-Cys(NBzl)-OH was coupled manually using 2.5 equiv. AA/HATU and 5 equiv. DIEA for 2 h. The precipitated crude peptide was dissolved in 50% acetonitrile/water and lyophilized. The HPLC analysis was carried out on a C4 analytical column using a gradient of 10-35% B over 30 min. For preparative HPLC, C4 column in gradient of 20-60% B was used to provide the peptide in ˜50% yield.

### Syntheses of Linear Protected Conotoxin-mr3e Analogues

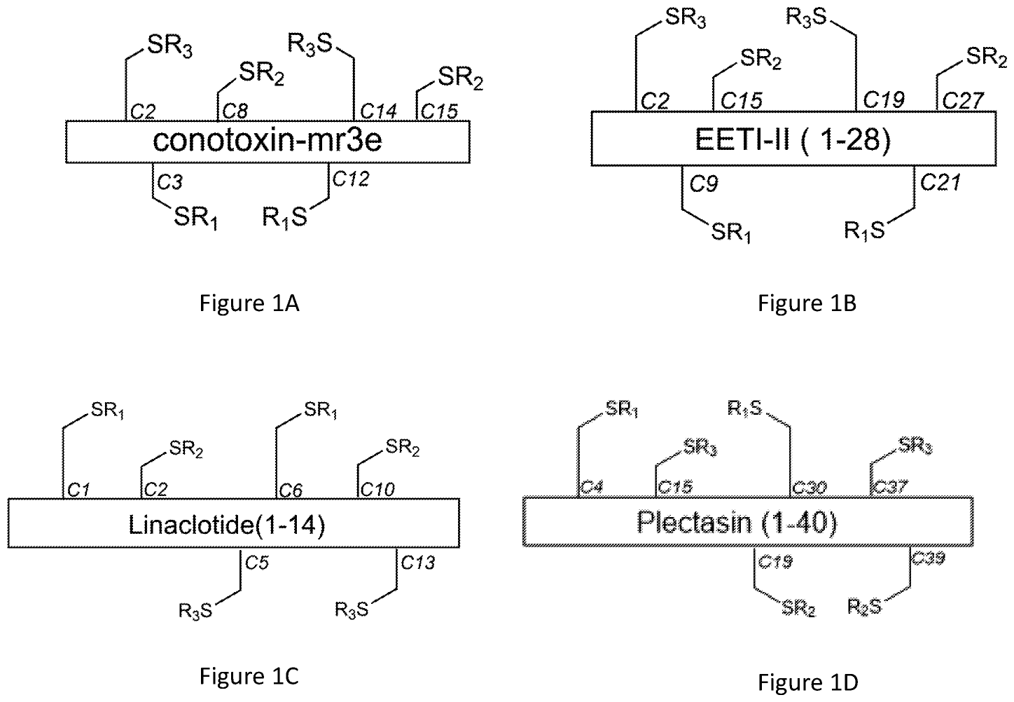

The synthesis of three analogues of the Conotoxin mr3e (Table 2 and FIG. 1A) was carried out using Fmoc-SPPS on a Rink amide resin (0.26 mmol/g, 0.2 mmol scale). Peptide synthesis was performed on peptide synthesizer and cleavage from the resin as described above. The precipitated crude peptide was dissolved in 50% acetonitrile/water and lyophilized. The HPLC analysis was carried out on a C4 analytical column using a gradient of 10-35% B over 30 min. For preparative HPLC, C4 column in gradient of 0-45% B was used to provide the peptide in ˜50% yield.

TABLE 2
Protecting groups (PGs) positions in
the prepared conotoxin-mr3e analogues

| Analogue | R₁ | R₂ | R₃ |
|---|---|---|---|
| 1 | H | NBzl | Acm |
| 2 | H | Acm | NBzl |
| 3 | NBzl | H | Acm |

### Syntheses of Linear Protected EETI-II Analogues

The synthesis of the six analogues of the EETI-II (Table 3 and FIG. 1B) was carried out using Fmoc-SPPS on a Rink amide resin (0.26 mmol/g, 0.2 mmol scale). Peptide synthesis was performed on peptide synthesizer and cleavage from the resin as described above. Fmoc-Asp(Boc)-Ser(ψMe,MePro)-OH and Fmoc-Cys(NBzl)-OH was coupled manually using 2.5 equiv. AA/HATU and 5 equiv. DIEA for 2 h. The precipitated crude peptide was dissolved in 50% acetonitrile/water and lyophilized. The HPLC analysis was carried out on a C4 analytical column using a gradient of 10-35% B over 30 min. For preparative HPLC, C4 column in gradient of 20-60% B was used to provide the peptide in ˜50% yield.

TABLE 3
PGs positions in the prepared EETI-II analogues

| Analogue | R₁ | R₂ | R₃ |
|---|---|---|---|
| 1 | H | NBzl | Acm |
| 2 | NBzl | H | Acm |
| 3 | NBzl | Acm | H |
| 4 | Acm | NBzl | H |
| 5 | Acm | H | NBzl |
| 6 | H | Acm | NBzl |

### Synthesis of Linear Protected Linaclotide Analogues

The synthesis was carried out using Fmoc-SPPS on 2-chlorotrityl chloride (Table 4 and FIG. 1C) resin (0.26 mmol/g, 0.2 mmol scale). Peptide synthesis was performed on peptide synthesizer and cleavage from the resin as described above. Fmoc-Cys(NBzl)-OH was coupled manually using 2.5 equiv. AA/HATU and 5 equiv. DIEA for 2 h. The precipitated crude peptide was dissolved in 50% acetonitrile/water and lyophilized. The HPLC analysis was carried out on a C4 analytical column using a gradient of 0-60% B over 30 min. For preparative HPLC, C4 column in gradient of 20-60% B was used to provide the peptide in ˜50% yield.

TABLE 4
PGs positions in the prepared linaclotide analogues

| Analogue | R₁ | R₂ | R₃ |
|---|---|---|---|
| 1 | H | Acm | NBzl |
| 2 | H | NBzl | Acm |
| 3 | NBzl | H | Acm |
| 4 | Acm | NBzl | H |
| 5 | Acm | H | NBzl |
| 6 | NBzl | Acm | H |

### Synthesis of Linear Protected Plectasin Analogues

The synthesis of the different plectasin analogues (Table 4 and FIG. 1D) and all the mutated variants of plectasin was carried out using Fmoc-SPPS on a Rink amide resin (0.26 mmol/g, 0.2 mmol scale). Peptide synthesis was performed on peptide synthesizer and cleavage from the resin as described above. Fmoc-Cys(NBzl)-OH in the different positions was coupled manually using 2.5 equiv. AA/HATU and 5 equiv. DIEA for 2 h. The precipitated crude peptide was dissolved in 50% acetonitrile/water and lyophilized. The HPLC analysis was carried out on a C18 analytical column using a gradient of 10-35% B over 30 min. For preparative HPLC, C18 column in gradient of 20-60% B was used to provide the peptide ˜45% yield.

TABLE 5
PGs positions in the prepared plectasin analogues

| Analogue | R₁ | R₂ | R₃ |
|---|---|---|---|
| 1 | H | NBzl | Acm |
| 2 | NBzl | H | Acm |
| 3 | NBzl | Acm | H |
| 4 | Acm | NBzl | H |
| 5 | Acm | H | NBzl |
| 6 | H | Acm | NBzl |

### Minimum Inhibitory Concentration (MIC) Assay

Fresh overnight colonies of methicillin resistant Staphylococcus aureus (MRSA) were suspended in 5 mL LB medium and were cultured overnight at 37° C. The bacterial suspension was adjusted to match 0.5 McFarland standards to obtain ˜1×10⁶ colony forming units (CFU) per mL. The synthetic proteins or trimethoprim (TMP) were serially diluted with LB in a 96-well plate (Greiner Bio-One) and the diluted bacterial suspension was added at 1:1 v/v ratio. The plate was covered with a breathing tape (AeraSeal™), incubated at 37° C. in ambient air for 17 h followed by absorbance readings at 600 nm to measure bacterial growth. Measurements were performed in triplicate. MIC values were determined as the lowest concentration of the compound by which no significant growth was observed.

The following stock solutions were prepared: (#1) 3 mg PdCl₂ was dissolved in 100 μl (170 mM) 6 M Gn·HCl buffer, pH 7. (#2) 2.5 mg DSF was dissolved in 100 μl (170 mM) ACN. (#3) 30 mg DTC was dissolved in 100 μl (1 M) H₂O. (#4) 3 mg GSH was dissolved in 100 μl (33 mM) H₂O.

### α-Conotoxin SI Synthesis

The lyophilized conotoxin peptide (0.5 mg, 0.3 μmol) was dissolved in 656 μl (0.5 mM) 6 M Gn·HCl buffer, pH 7, and treated with 10 equiv. DSF (25 μl from stock) for 10 s at 37° C. Subsequently, the pH of the reaction was adjusted to 1 using 0.1 M HCl and 10 equiv. PdCl₂ (16 μl from stock #1) was added for 5 min at 37° C. Then, 30 equiv. (8 μl from stock #3) DTC followed by 10 equiv. DSF were added. pH adjustment to 7 and incubation at 37° C. for 10 s, followed by exposure to UV irradiation at 350 nm for 8 min at room temperature immediately afforded the native α-conotoxin SI. In the second synthetic approach, the lyophilized conotoxin peptide (0.5 mg, 0.3 μmol) was also dissolved in 656 μl (0.5 mM) 6 M Gn·HCl buffer, pH 7, and treated with 10 equiv. DSF (25 μl from stock) for 10 s at 37° C. followed by exposure to UV irradiation at 350 nm for 8 min at room temperature Subsequently, the pH of the reaction was adjusted to 1 using 0.1 M HCl and 15 equiv. PdCl₂ (25 μl from stock #1) was added for 5 min at 37° C. Then, 30 equiv. (8 μl from stock #3) DTC followed by 10 equiv. DSF were added. pH adjustment to 7 and incubation at 37° C. for 10 s immediately afforded the native α-conotoxin SI.

### Conotoxin-mr3e Synthesis

The lyophilized Conotoxin-mr3e peptide (0.5 mg, 0.1 μmol) was dissolved in 462 μl 6 M Gn·HCl buffer, pH 7 (0.5 mM), and treated with 10 equiv. DSF (20 μl from stock #2) was added for 10 s followed by exposure to UV irradiation at 350 nm for 8 min at room temperature. Subsequently the pH of the reaction was adjusted to 1 using 0.1 M HCl and 10 equiv. PdCl₂ (13 μl from stock #1) was added for 5 min at 37° C. Then, 30 equiv. (6 μl from stock) DTC followed by in situ addition of 10 equiv. DSF, pH adjustment to 7 and incubation at 37° C. for 10 s afforded the native Conotoxin-mr3e.

### EETI-II Synthesis

The lyophilized EETI-II peptide (0.5 mg, 0.1 μmol) was dissolved in 303 μl 6 M Gn·HCl buffer, pH 7 (0.5 mM), and treated with 10 equiv. DSF (14 μl from stock #2) was added for 10 s followed by exposure to UV irradiation at 350 nm for 8 min at room temperature. Subsequently the pH of the reaction was adjusted to 1 using 0.1 M HCl and 10 equiv. PdCl₂ (9 μl from stock #1) was added for 5 min at 37° C. Then, 30 equiv. (4 μl from stock) DTC followed by in situ addition of 10 equiv. DSF, pH adjustment to 7 and incubation at 37° C. for 10 s afforded the native EETI-II in 32% isolated yield.

### Oxidative Folding of Unprotected EETI-II

Purified EETI-II was dissolved in sodium phosphate buffer (0.2 M, pH 8) and oxidized by stirring in open air for 72 h. The product was isolated in 28% yield, in comparison to 13 min and 32% isolated yield using the inventors approach.

### Linaclotide Synthesis

The lyophilized linaclotide peptide (0.5 mg, 0.2 μmol) was dissolved in 515 μl 6 M Gn·HCl buffer, pH 7, (0.5 mM) and treated with 10 equiv. DSF (15 μl from stock #2) for 10 s followed by exposure to UV irradiation at 350 nm for 8 min at room temperature. Subsequently the pH of the reaction was adjusted to 1 by 0.1 M HCl and 15 equiv. PdCl₂ (23 μl from stock #1) was added for 5 min at 37° C. Then, 30 equiv. of DTC (7 μl from stock #3) followed by 2 equiv. GSH 10 equiv. (16 μl from stock #5) and 10 equiv. DSF were added in situ. pH adjustment to 7 and incubation at 37° C. for 10 s immediately afforded the native linaclotide. The addition of 2 equiv. of GSH was found to facilitate the recovery of the peptide from the bounded Pd residues.

### Plectasin Synthesis

The lyophilized plectasin peptide (0.5 mg, 0.1 μmol) was dissolved in 208 μl (0.5 mM) 6 M Gn·HCl buffer, pH 7, and treated with 10 equiv. DSF (9 μl from stock #2) for 10 s followed by exposure to UV irradiation at 350 nm for 8 min at room temperature. Subsequently the pH of the reaction was adjusted to 1 by 0.1 M HCl and 10 equiv. PdCl₂ (6 μl from stock #1) was added for 5 min at 37° C. Then, 30 equiv. (3 μl from stock #3) DTC was added followed by in situ addition of 5 equiv. GSH (5 μl from stock #4) and 10 equiv. DSF. pH adjustment to 7 and incubation at 37° C. for 10 s immediately afforded the native plectasin. The addition of 5 equiv. GSH was found to facilitate the recovery of the peptide from the bound Pd residues.

### Synthesis of Protected RANTES Linear Peptide

The synthesis was carried out using Fmoc-SPPS on a Rink amide resin (0.26 mmol/g, 0.2 mmol scale). Peptide synthesis was performed on peptide synthesizer and cleavage from the resin as described above. Fmoc-Asp(Boc)-Thr(ψMe,MePro)-OH, Fmoc-Tyr(Trt)-Thr(ψMe,MePro)-OH and Fmoc-Val-Thr(ψMe,MePro)-OH was coupled manually using 2.5 equiv. AA/HATU and 5 equiv. DIEA for 2 h. The precipitated crude peptide was dissolved in 50% acetonitrile/water and lyophilized. The HPLC analysis was carried out on a C4 analytical column using a gradient of 0-60% B over 30 min. For preparative HPLC, C4 column in gradient of 20-60% B was used to provide the peptide in ˜50% yield.

### CD Spectroscopy of Purified α-Conotoxin-SI

The peptide was dissolved in sodium acetate buffer (0.35 mg/ml) at pH 7. The exact concentration of the protein solution was determined by Pierce® BCA Protein Assay Kit, Thermo scientific. With this solution circular dichroism spectrum was recorded in a Chirascan (Applied Photophysics) instrument.

### CD Spectroscopy of Purified RANTES

The peptide was dissolved in phosphate-buffered saline (PBS) (1.58 mg/ml) at pH 7. The exact concentration of the protein solution was determined by Pierce® BCA Protein Assay Kit, Thermo scientific. With this solution circular dichroism spectrum was recorded in a Chirascan (Applied Photophysics) instrument.

### CD Spectroscopy of Purified Plectasin

The peptide was dissolved in water (0.37 mg/ml) at pH 7. The exact concentration of the protein solution was determined by Pierce® BCA Protein Assay Kit, Thermo scientific. The circular dichroism spectrum was recorded in a Chirascan (Applied Photophysics) instrument.

### CD Spectroscopy of Purified EETI-II

The peptide was dissolved in phosphate-buffered saline (PBS) (3.33 mg/ml) at pH 7. The exact concentration of the protein solution was determined by Pierce® BCA Protein Assay Kit, Thermo scientific. The circular dichroism spectrum was recorded in a Chirascan (Applied Photophysics) instrument.

### Ultrafast One-Pot Two Disulfide Bonds Formation in RANTES

The lyophilized RANTES peptide (0.5 mg, 0.06 nmol) was dissolved in 125 μl (0.5 mM) 6 M Gn·HCl buffer, pH 7, and treated with 10 equiv. DSF (4 μl from stock) for 1 s at 37° C. Subsequently, the pH of the reaction was adjusted to 1 by 0.1 M HCl and 15 equiv. PdCl₂ (6 μl from stock) was added for 5 min at 37° C. Then, 30 equiv. (2 μl from stock) DTC followed by 10 equiv. were added. Immediately after the pH was readjusted to 7 and put in 37° C. for 1 s to afford the native RANTES.

### Example 1

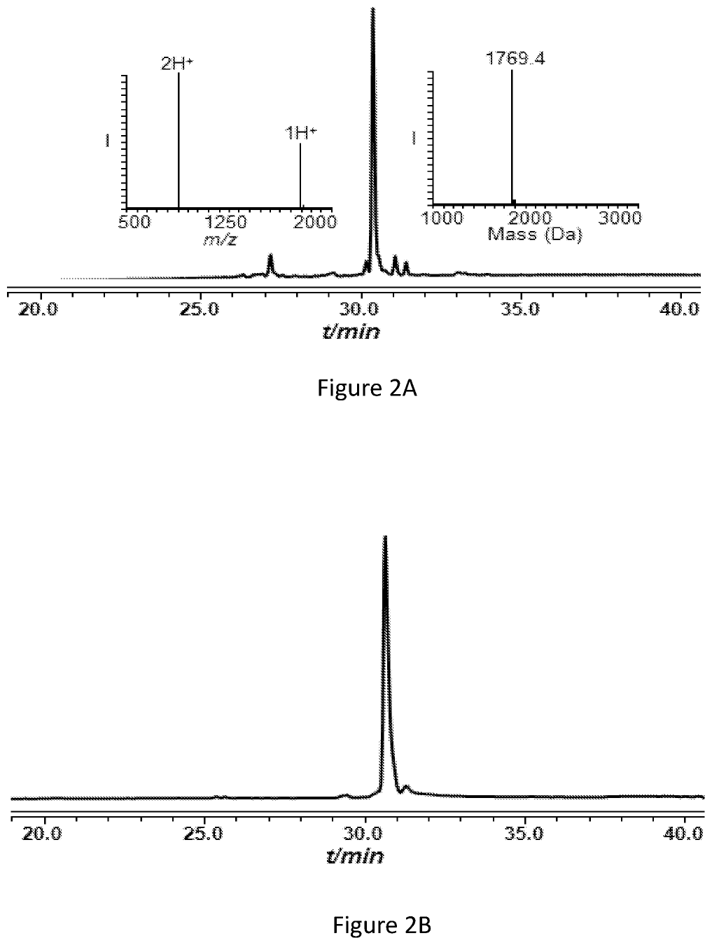

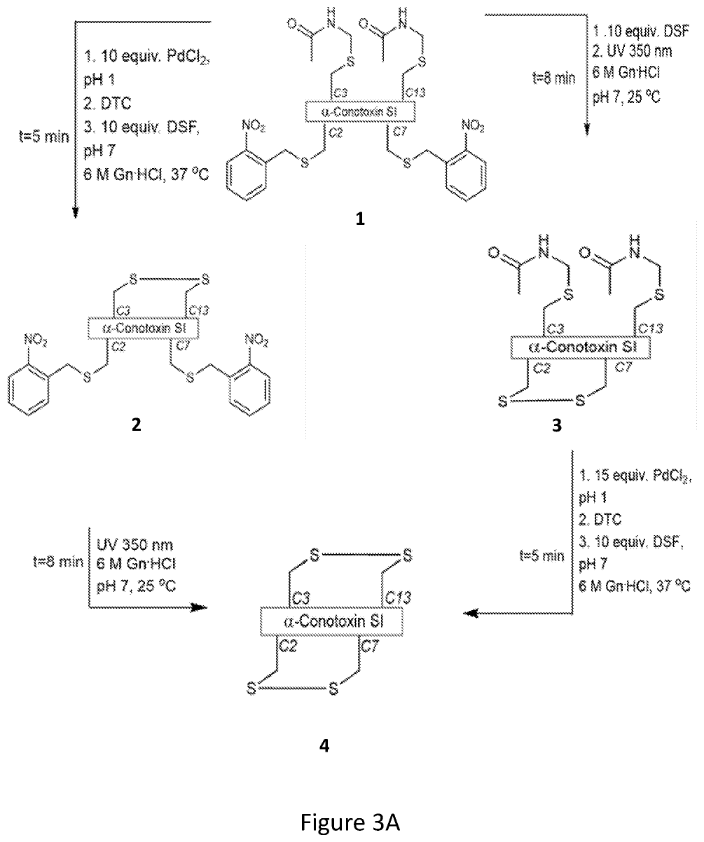

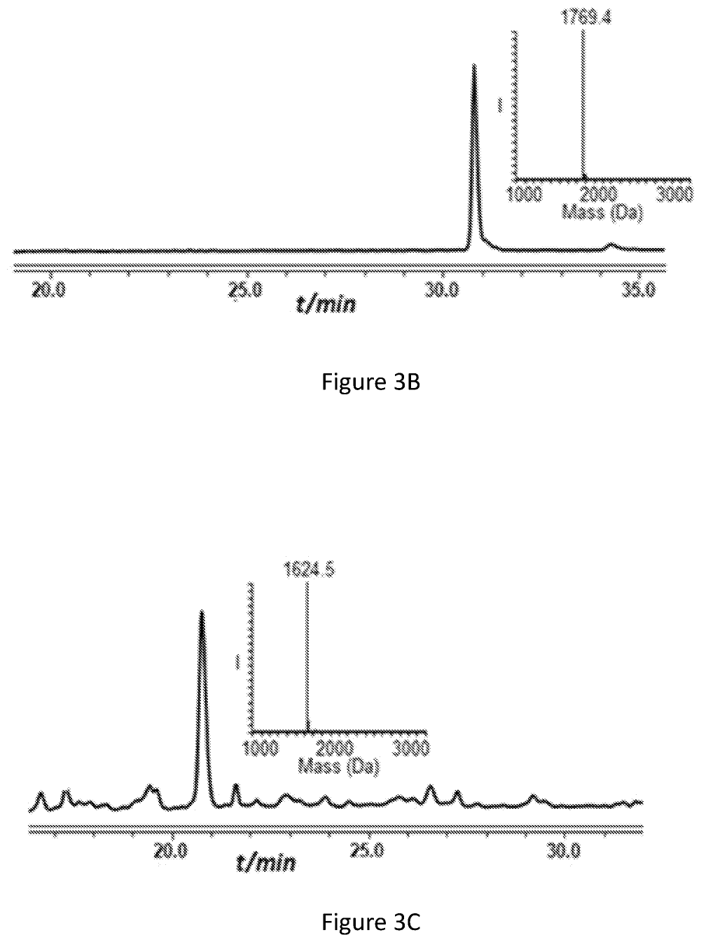

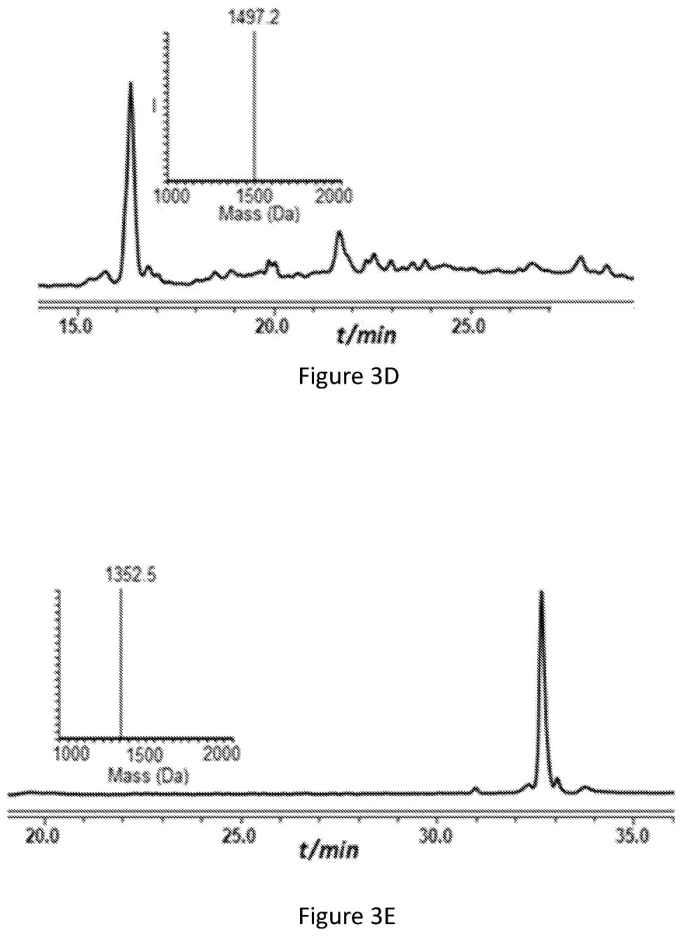

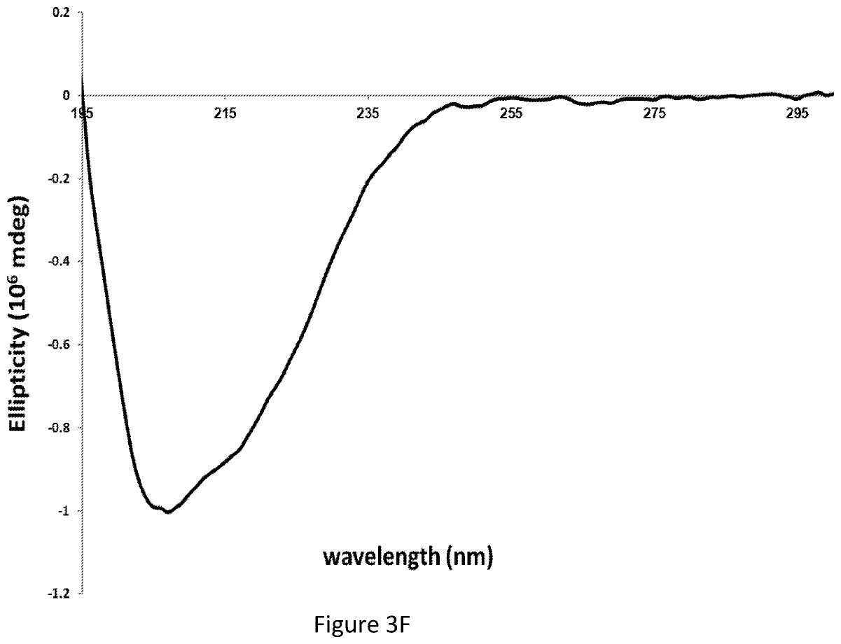

The inventors have shown the efficient synthesis of various targets such as the α-conotoxin SI peptide, composed of 13 AAs and two disulfide bonds. In order to probe the effect of the order of disulfide bonds formation in this system, the inventors prepared via Fmoc-solid phase peptide synthesis (SPPS), α-conotoxin peptide, bearing two Cys (3&13) protected with Acm, and the two Cys (2&7) protected with 2-nitrobenzyl (NBzl), (FIGS. 2A-B). The inventors dissolved the peptide in 6 M GnHCl and exposed it to 10 equiv. PdCl₂ for 5 min in pH 1, at 37° C. Subsequently, the inventors added 20 equiv. diethyldithiocarbamate (DTC) to quench and precipitate Pd. The inventors then added 10 equiv. DSF and adjusted the pH to 7, to get the immediate formation of the first disulfide bond between Cys (3&13), as detected by HPLC-MS (FIG. 3C). Exposure of this reaction mixture to UV irradiation at 350 nm (24 watts, ˜3×10¹⁶ sec/cm³ photons) in a photochemical 16 chamber reactor under cooling to room temperature, for 8 min, furnished quantitatively the second disulfide bond between Cys (2&7) (FIG. 3A and FIG. 3E).

The inventors then sought to probe the effect of reversing the orthogonal use of Pd and UV. Therefore, the inventors first exposed the peptide to UV irradiation in presence of 10 equiv. DSF, for 8 min, followed by treatment with 15 equiv. PdCl₂ under the mentioned above conditions, (FIG. 3A and FIGS. 3D-E). Such an operation furnished the desired oxidized product in both regio- and chemoselective manner mediated by Pd^{II} deprotection of Cys (Acm) in presence of S—S bond or Cys (NBzl) (FIG. 3E and FIG. 3F).

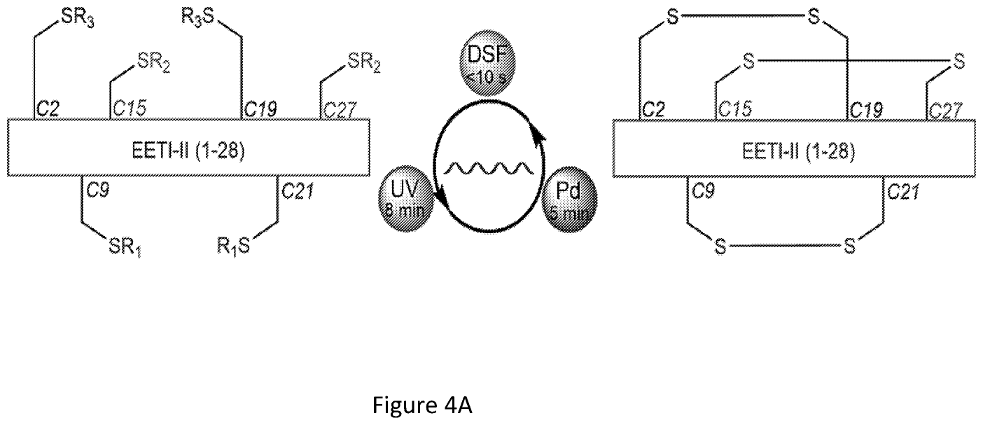

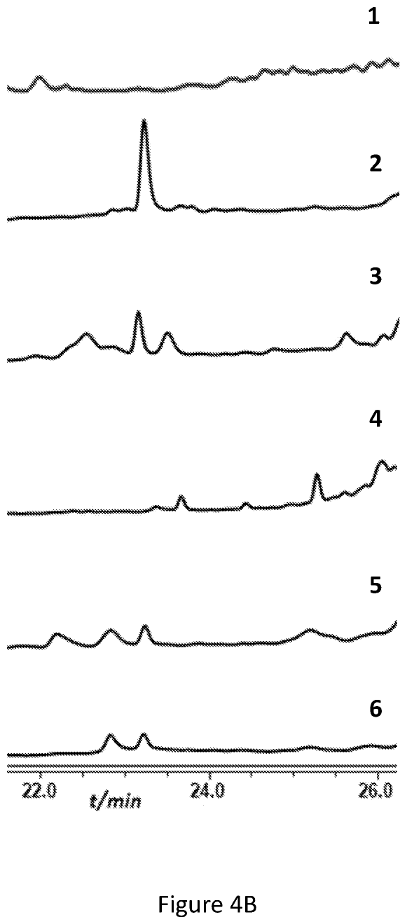

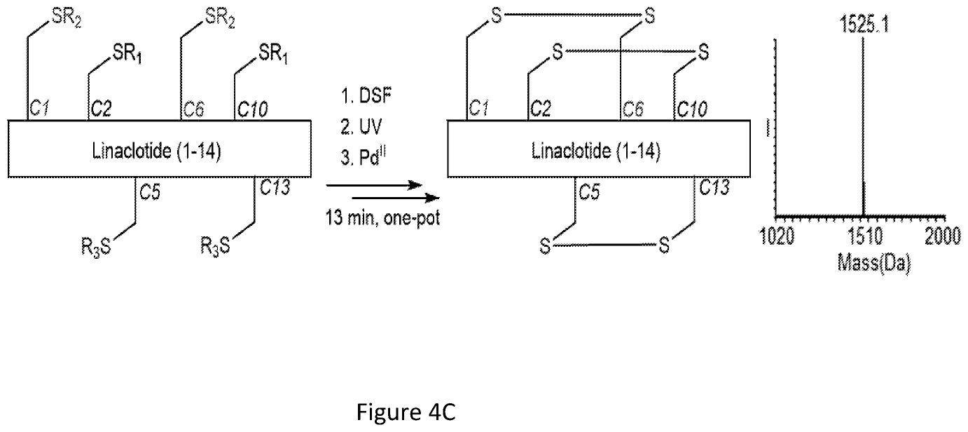

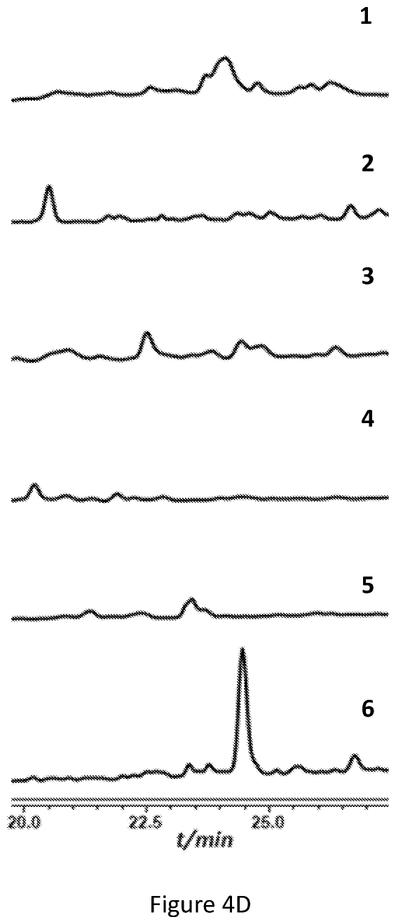

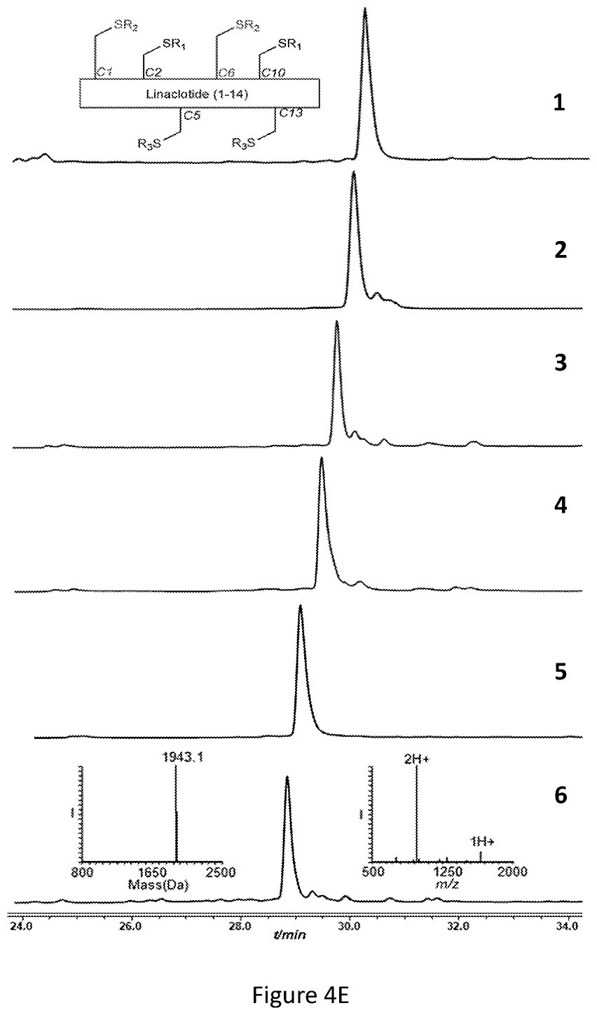

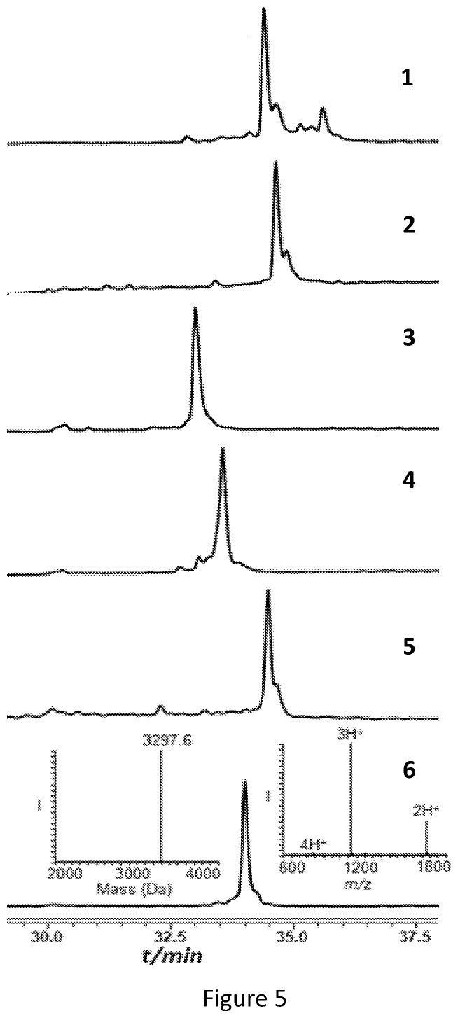

With this observed synthetic flexibility in two disulfide bonds formation, the inventors decided to examine the synthetic design for a more challenging example, specifically from the knotted protein family bearing three disulfide bonds. In the knot motif the peptide backbone extensively folded back on itself and by cross bracing of the three disulfide bonds. This unique architecture has an exceptional effect on its chemo, mechano and proteolytic stability. As an example the inventors choose the ecballium elaterium trypsin inhibitor II (EETI-II) mini-protein, a highly potent trypsin inhibitor, composed of 28-residues and bearing three disulfide bonds between Cys (9&21), Cys (15&27) and Cys (2&19). The inventors synthesized all six possible analogues in which in each analogue a pair of Cys were kept free, the second pair Cys were protected with Acm while the third with NBzl. In contrast to what was observed in the case of two disulfide bonds, changing the order of disulfide bonds formation affected dramatically the EETI-II synthesis (FIGS. 4A-B). Remarkably, only one analogue among all the six possible forms, with Acm at position 2&19 and NBzl at position 15&27, enabled the inventors to produce the fully oxidized EETI-II in 32% isolated yield (FIG. 4B, 2). Moreover, only one synthetic strategy was successful in which UV light irradiation has been applied prior to Pd mediated Acm removal (FIG. 5). In all other possibilities where different order of disulfide bonds formation was applied, the inventors observed complicated HPLC chromatograms, possibly due to peptide solubility problems of the formed intermediates or reshuffling, despite forming the correct disulfide bond (FIG. 4B). Notably, beside the 15 possible regioisomers of fully oxidized EETI-II, an additional 60 intermediates can be produced during uncontrolled disulfide rearrangement. A similar pattern was detected in the structurally rigid 14 AAs peptide drug linaclotide (FIGS. 4C-E), in which only one analogue among the six could be utilized for the successful synthesis.

It is well established that in knotted proteins there is a strong mechanical stress on the disulfide bonds due to intrinsic torsional strain. In principle, formation of a disulfide within a protein fold could force the dihedral angles of both the S—S bond and the Cys side chains into strained conformations that could generate internal strain that is strong enough to tear the disulfide bond. Interestingly, the successful synthetic order in this study, perfectly matches the previously elucidated order of disulfide bonds formation in EETI-II during natural folding process. The inventors therefore hypothesize, when forming the wrong intermediate this could lead to internal mechanical forces that lead to disulfide cleavage or/and unfavored structures that has low solubility. The natural disulfide bonds order within the protein folding mechanism is perhaps resulting in more mechano-stable intermediates while avoiding disulfide undesired rearrangement. A complete unfolding of the polypeptide should in principle reduce dramatically such a massive mechanoforces during the synthetic process, however, in the case of knotted proteins it was reported that noticeable structural features remain stable also in presence of strong denaturing conditions (e.g. 6 M GnHCl).

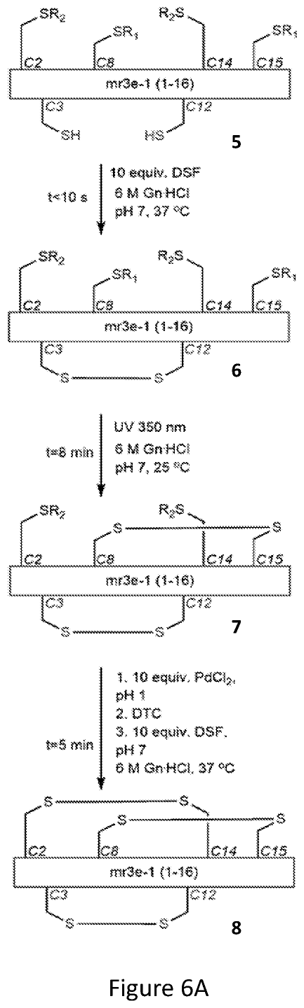

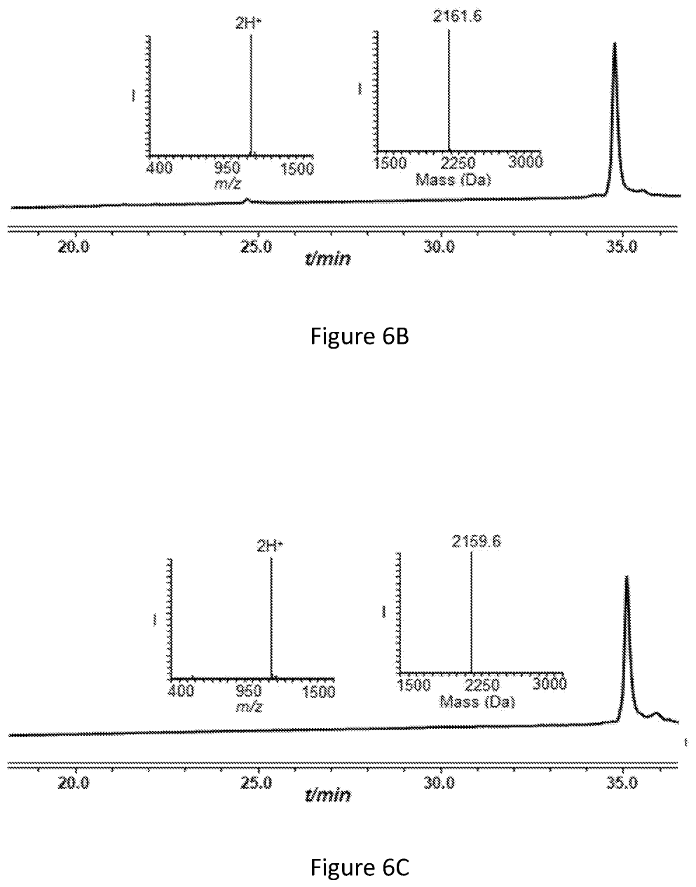

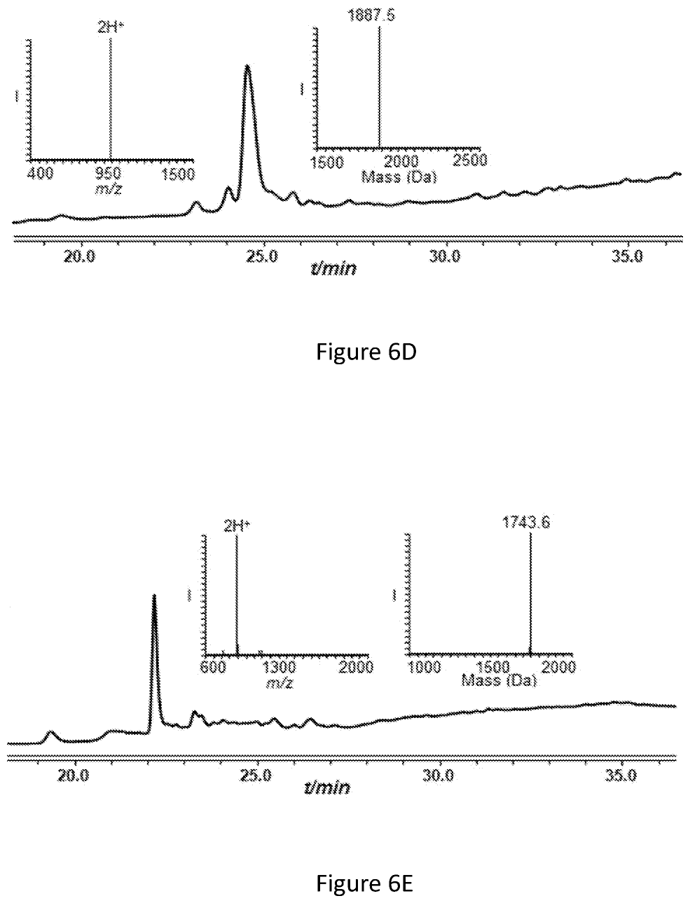

Based on these findings, the inventors envisioned that peptides and proteins, lack a knotted structural motif, and therefore known to undergo denaturation under the present synthetic conditions, may not be restricted to one particular order. To examine this notion, the inventors chose the conotoxin mr3e (mr3e) composed of 16 AAs and three disulfide bonds as a model peptide, which is known to have great solubility and structural flexibility. The inventors first synthesized the mr3e peptide bearing the free Cys pair at positions 3&12, NBzl protected at Cys 8&15 and the Acm protected at Cys 2&14. Applying the ultrafast synthetic strategy using DSF followed by exposure to UV and subsequent treatment with Pd^{II}, gave within 13 min a single product (among the 15 possible structural isomers) as detected by HPLC-MS analyses (FIGS. 6A-E). The inventors then made another two analogues of differently protected mr3e, to examine the effect of disulfide bonds formation order. Upon subjecting these analogues to the exact same process, a perfectly matched single product was detected via HPLC-MS.

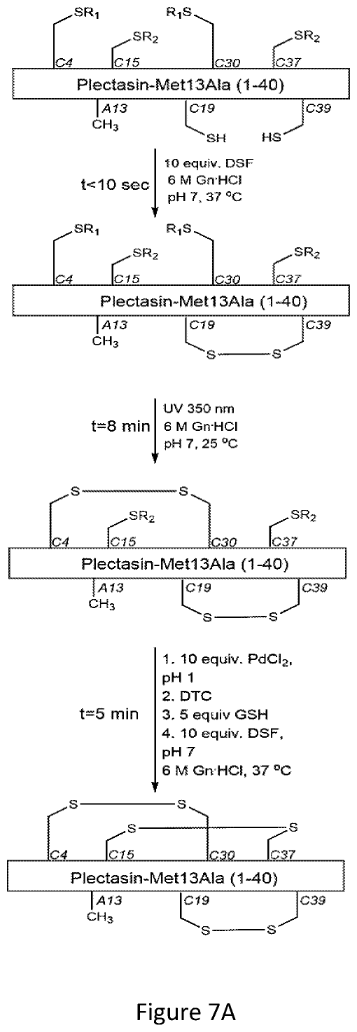

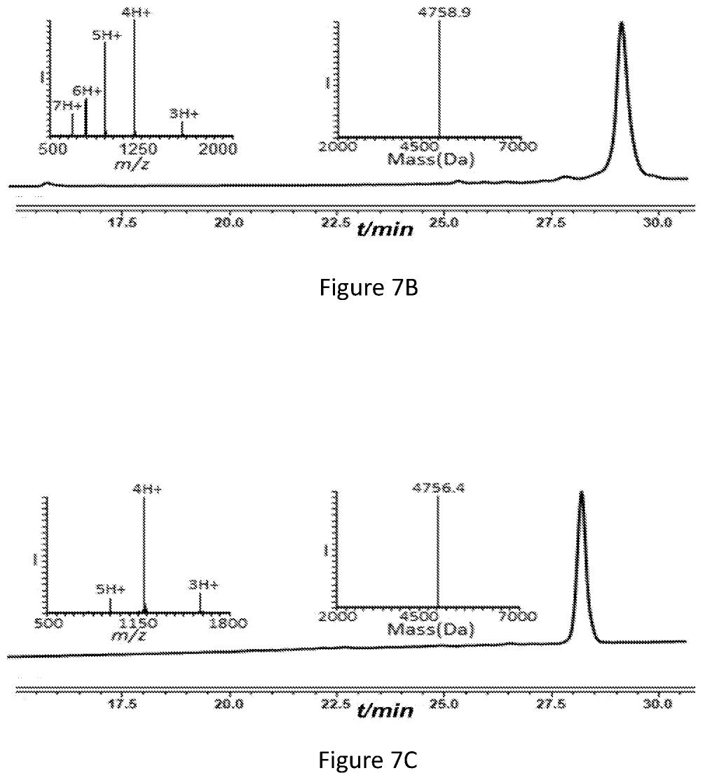

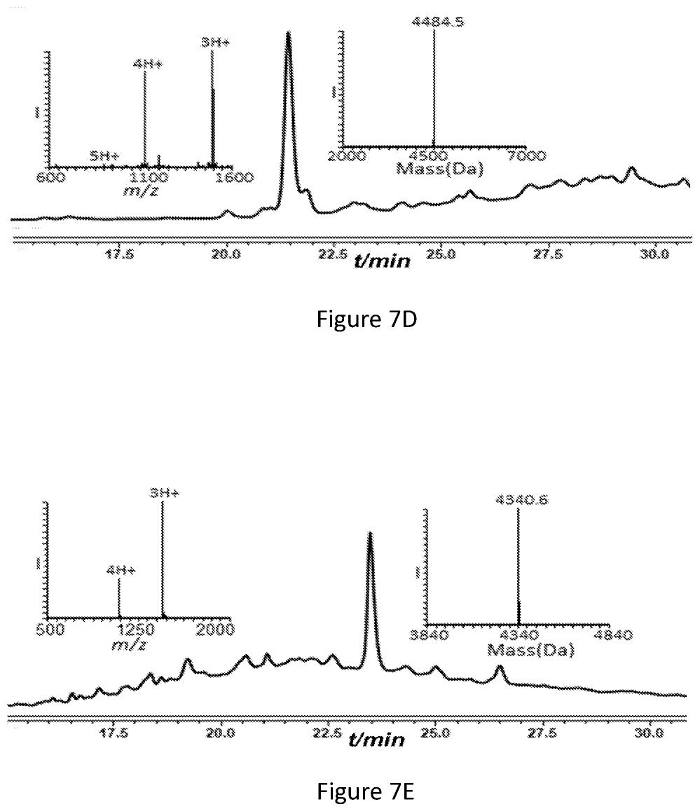

To further evaluate the results, the inventors chose plectasin, a natural AMP discovered in fungi and has significant potency against drug-resistant Gram-positive bacteria. Plectasin is composed of 40 AAs with three disulfide bonds between Cys (4&30), Cys (19&39) and Cys (15&37). The inventors assembled plectasin analogue via SPPS protocol, bearing two free Cys (4&30), two Cys protected with NBzl (19&39), two Cys protected with Acm (15&37). Upon subjecting the linear peptide to the synthetic design, a single product was formed within 13 min and was further characterized to ensure the generation of natural isomer. For this study, in order to investigate the effect of disulfide bonds order formation, the inventors synthesized all the six possible analogues and tested them applying the presented synthetic strategy (FIGS. 7A-E). Each peptide was dissolved in 6 M Gn·HCl buffer, pH 7 and exposed to 10 equiv. DSF in 37° C., followed by 8 min UV irradiation in 25° C. Subsequently, the reaction mixture was treated with 10 equiv. Pd^{II} for 5 min under pH 1, at 37° C. and then quenched with 20 equiv. DTC and 5 equiv. glutathione (GSH). Immediately after, the third disulfide bond was accomplished in situ upon the addition of 10 equiv. DSF and pH readjustment to 7. The use of GSH as an additional Pd scavenger to DTC, was found to further enhance the release of S—Pd bond between the peptide and Pd^{II} traces, as treatment with DTC only was not sufficient. All six analogues provided the desired product in very good yields (˜40%). The inventors' findings regarding the different order in plectasin synthesis also supported the inventors' notion about the important role of structure flexibility of the formed intermediates, which assist in avoiding disulfide bonds reshuffling and their high solubility in the reaction media.

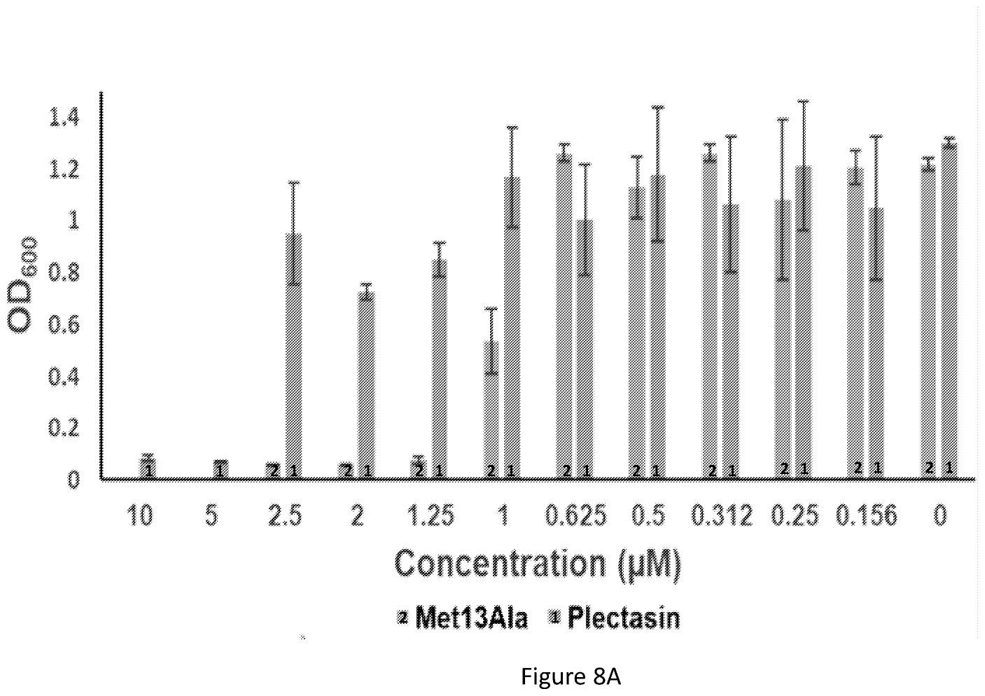

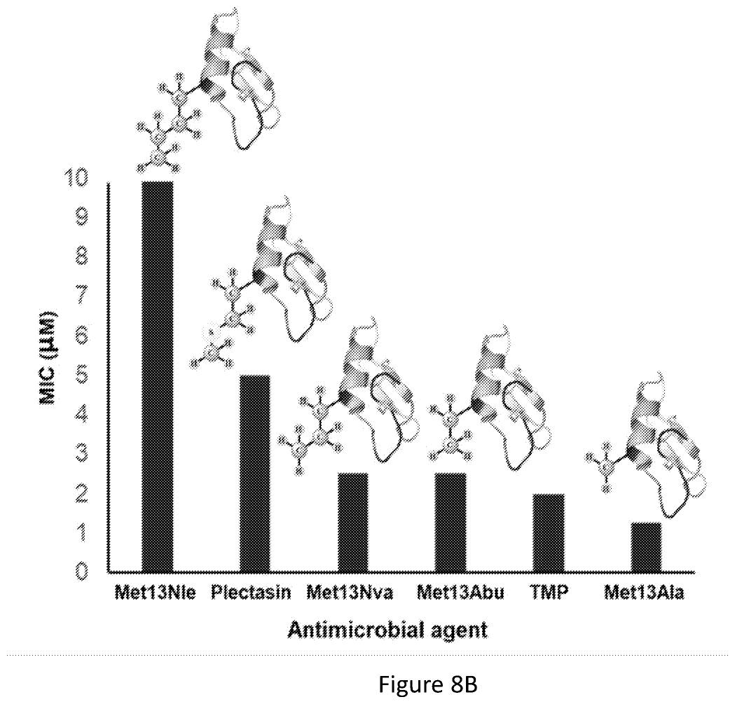

These findings prompted the inventors to attempt and improve the antimicrobial activity of plectasin applying a single point mutation study in its sequence, without the need for tedious prior synthetic evaluations for each derivative. For this goal, the inventors chose position 13 (naturally occurring as methionine (Met)), which although is not involved directly in the binding with the bacterial cell wall precursor lipid-II, has been reported to be a sensitive residue in terms of effect on the total antimicrobial activity in a yet unknown mechanism. The following mutations have been implemented in the plectasin peptides sequence: Met13Norleucine (Nle), Met13Norvaline (Nva), Met13Amino butyric acid (Abu) and Met13Alanine (Ala). All these derivatives were synthesized via Fmoc-SPPS and subjected to the presented ultrafast disulfide bonds formation followed by testing against methicillin resistant Staphylococcus aureus (MRSA) (FIG. 8A). Excitingly, the mutation of Met to Ala resulted in a four-fold improvement of the antimicrobial activity even in comparison to the clinically widely used antibiotic trimethoprim (TMP), as can be judged by minimum inhibitory concentration (MIC) analysis (FIG. 8B).

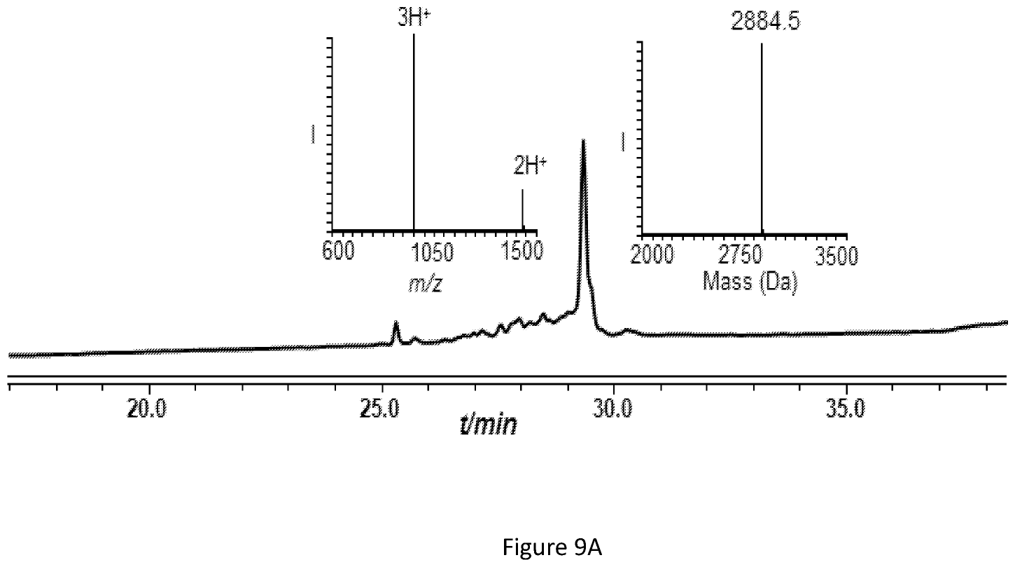

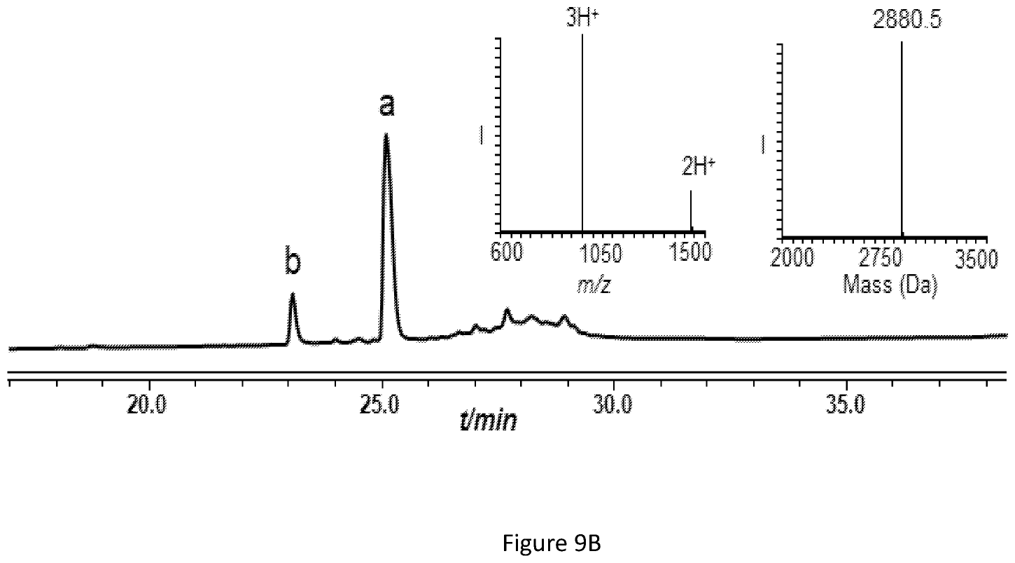

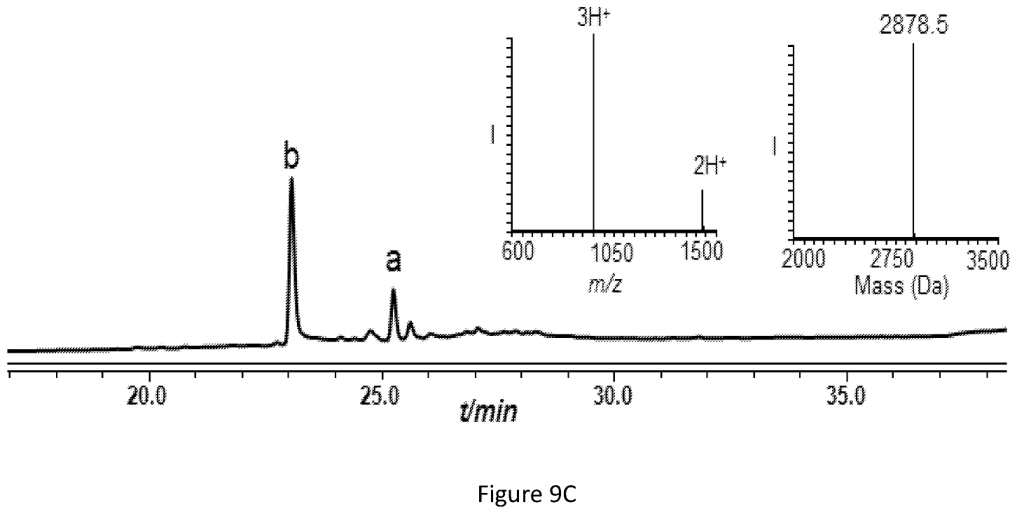

In summary, the inventors have investigated the order of disulfide bonds formation as an important parameter in the presented de novo synthetic strategy for the ultrafast formation of multiple disulfide bonds. The current study revealed interesting behavior of EETI-II from the knotted protein family, in which a possible internal mechanoforces could mediate disulfide bonds cleavage and reshuffling when non preferable synthetic order is applied. This finding could be attributed to the relative structural stability of these complicate yet fascinating miniproteins, even under strong denaturation conditions. The inventor's findings could inspire studies based on mechanochemistry and structural biology tools in order to probe yet unknown possible correlation between mechano-stability of peptide intermediates and the native protein folding pathway. When comparing the synthesis of EETI-II using the inventors strategy and oxidative folding approach, the isolated yields were similar (˜30%), however, the required time using the inventors approach was 13 min compared to 3 days for the oxidative approach (FIGS. 9A-C). Taking advantages of the current approach, the inventors were able to straightforwardly design, prepare and study various plectasin AMP unnatural derivatives having a single point mutation at Met13. The inventors discovered a new analogue with four-fold enhanced activity (Met13Ala) relative to the wild type plectasin. The inventors anticipate this study will pave the way for more simplified synthesis of peptides and proteins libraries for various applications e.g. de novo protein design, drug developments and structure activity relationships studies.

### Example 2

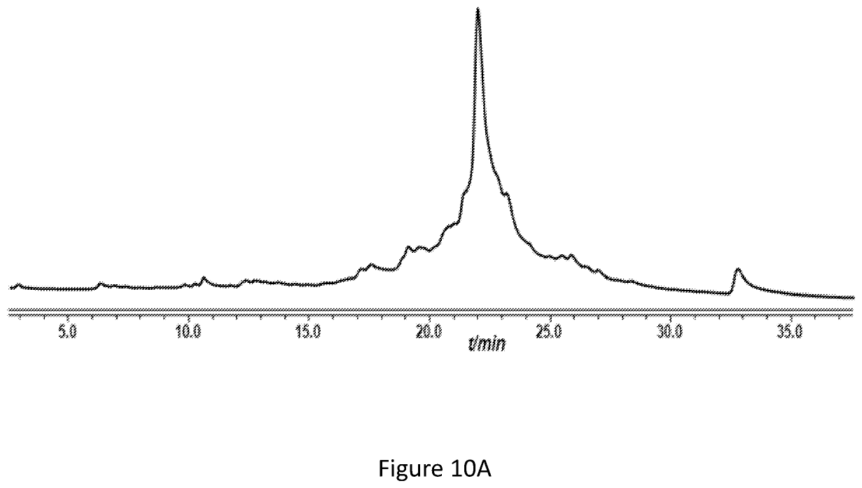

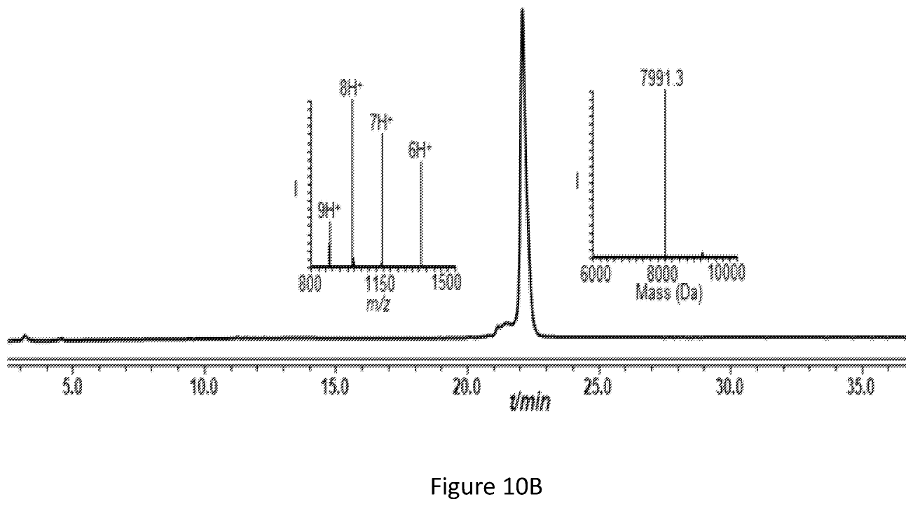

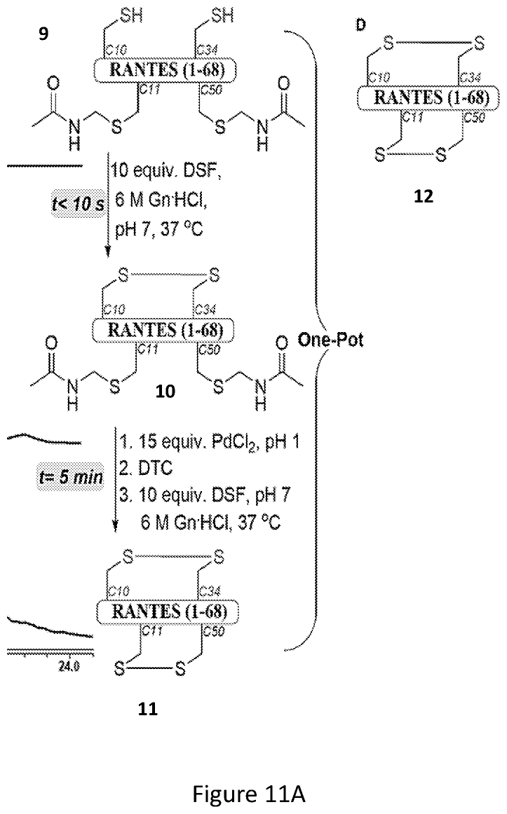

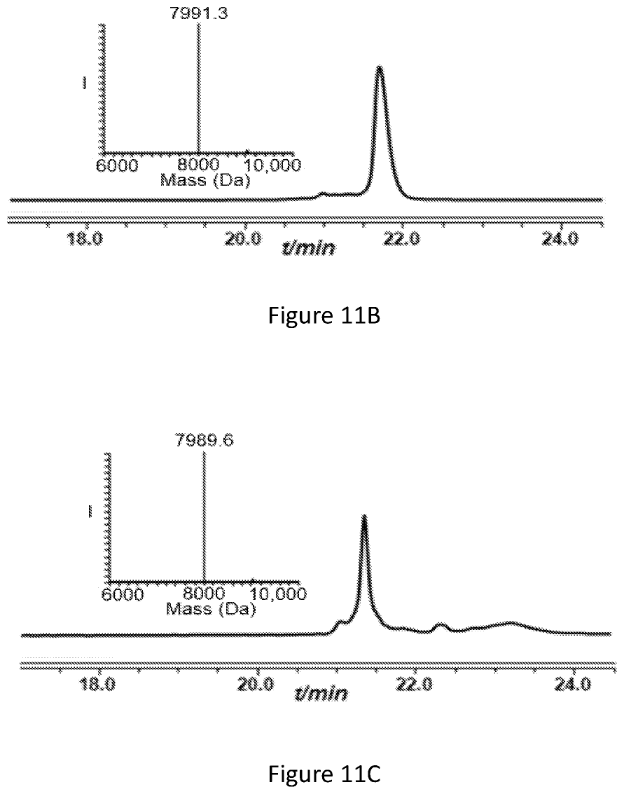

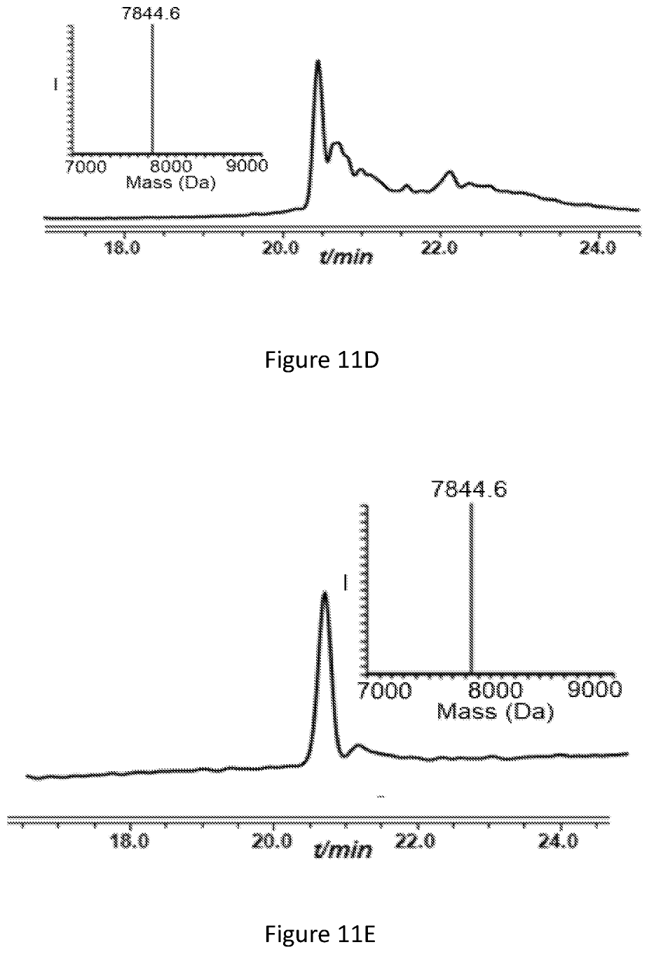

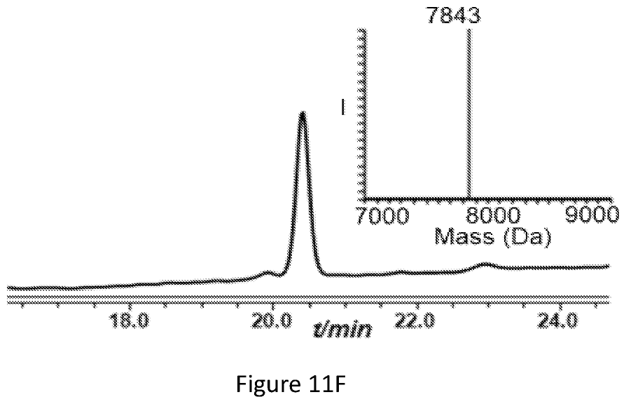

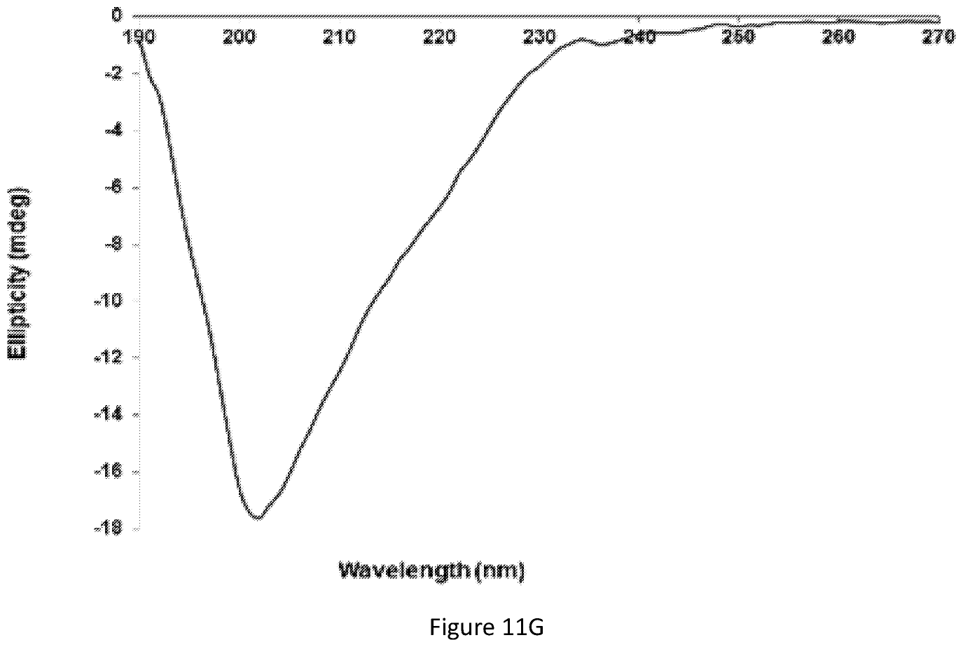

The inventors examined the presented set of conditions in more challenging synthetic systems. Therefore, the RANTES chemokine protein from the cytokine family that directs trafficking of leukocytes during inflammation, was chosen. The inventors prepared this protein, composed of 68 AAs and two disulfide bonds between Cys (10&34) and Cys (11&50), via SPPS with Cys 10 and 34 in the free form, and Cys 11 and 50 modified with the Acm (FIGS. 10A-B). Applying the inventors developed synthesis afforded the native protein with the correct disulfides within 5 min, which was isolated in 35% yield for the two oxidation steps (FIGS. 11A-D). The chromatographic retention time of the product matched perfectly with the commercially available protein and the CD signature of the correct isomer folding was detected (FIGS. 11E-G).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for forming a plurality of disulfide bonds, comprising
a. providing a peptide comprising (i) a deprotected cysteine pair, (ii) a first cysteine pair protected by a first protecting group, and optionally (iii) a second cysteine pair protected by a second protecting group;
b. adding to said peptide a sufficient amount of a reagent comprising a disulfide, a diselenide, a thiol, a sulfide, a thiocarbonate, a dithiocarbonate, thiocarbamate, or a dithiocarbamate, including any combination, a disulfanyl derivate, a metal complex or a salt thereof, thereby forming a first disulfide bond between thiols of said deprotected cysteine pair;
c. providing said peptide under conditions sufficient for deprotecting the first protecting group and adding a sufficient amount of said reagent to said peptide, thereby forming a second disulfide bond between thiols of said first cysteine pair;
wherein said plurality of disulfide bonds comprises intramolecular bonds; and wherein the steps b and c are performed in one pot.

2. The method of claim 1, wherein the step b is performed prior to the step c or subsequent to the step c.

3. The method of claim 1, further comprising step d, comprising adding a sufficient amount of said reagent to said peptide, or to a reaction mixture comprising the peptide; and providing said peptide or said reaction mixture under conditions sufficient for deprotecting the second protecting group, thereby forming a second disulfide bond between thiols of said second cysteine pair; optionally wherein said step d is performed prior to the step c or subsequent to the step c; optionally wherein the steps b to d are performed in one pot.

4. (canceled)

5. (canceled)

6. The method of claim 1, wherein any one of said first protecting group and said second protecting group independently comprises a photocleavable protecting group or a photostable protecting group; wherein conditions sufficient for deprotecting said photostable protecting group comprise adding a sufficient amount of a transition metal catalyst to said peptide; optionally wherein said photocleavable protecting group comprises o-nitrobenzyl (NBzl); optionally wherein said photostable protecting group comprises acetamidomethyl; optionally wherein said transition metal catalyst comprises a transition metal, a complex or a salt thereof.

7. (canceled)

8. (canceled)

9. (canceled)

10. The method of claim 6, wherein said transition metal comprises a metal selected from the group consisting of Pt, Pd, Ru, Cu, Ni, Co, Ti, Zn and Ag or any combination thereof, and wherein the metal is in an elemental state or in an oxidized state; optionally wherein said transition metal catalyst comprises Pd, including any salt or a complex thereof.

11. (canceled)

12. (canceled)

13. The method of claim 6, further comprising adding an effective amount of a complexing agent to the peptide in contact with the transition metal catalyst; wherein the effective amount of the complexing agent is sufficient for substantially complexing the transition metal catalyst; optionally wherein the complexing agent comprises at least one of a thiol, a thiocarbonate, a dithiocarbonate, thiocarbamate, or a dithiocarbamate, including any disulfanyl derivate thereof; optionally wherein the complexing agent comprises glutathione.

14. (canceled)

15. (canceled)

16. (canceled)

17. The method of claim 6, wherein conditions sufficient for deprotecting said photocleavable protecting group comprises exposing said peptide to light for a time period sufficient for de-protection of said photocleavable protecting group; and wherein said light has a wavelength suitable for deprotecting said photocleavable protecting group.

18. (canceled)

19. The method of claim 17, wherein said wavelength is between 200 and 600 nm.

20. The method of claim 1, wherein said reagent is selected from the group consisting of Pd, Cu, sulfide, glutathione, disulfiram (DSF), and diethyldithiocarbamate (DTC), including any combination, any complex or a salt thereof.

21. A method for forming a disulfide bond, comprising:
a. providing a peptide comprising a first cysteine pair, wherein each thiol of said first cysteine pair is protected by a photocleavable protecting group;
b. exposing said peptide to light for a time period sufficient for substantially deprotecting said photocleavable protecting group;
c. adding a sufficient amount of a reagent comprising a disulfide, a diselenide, a thiol, a sulfide, a thiocarbonate, a dithiocarbonate, thiocarbamate, or a dithiocarbamate, including any combination, a disulfanyl derivate, a metal complex or a salt thereof, thereby forming a first disulfide bond between thiols of the first cysteine pair;
wherein said disulfide bond is an intramolecular bond; and wherein the step b and the step c are performed simultaneously or subsequently.

22. The method of claim 21, wherein said peptide further comprises an additional cysteine pair, wherein said additional cysteine pair is protected by a photostable protecting group; and wherein said method further comprising step d of (i) providing said peptide under conditions sufficient for substantially deprotecting said photostable protecting group; and (ii) adding a sufficient amount of the reagent thereby forming a second disulfide bond between thiols of the additional cysteine pair; and wherein conditions sufficient for substantially deprotecting said photostable protecting group comprise adding a sufficient amount of a transition metal catalyst to said peptide; wherein said step d is performed prior to the steps b and c or subsequent to the steps b and c; optionally wherein at least two of the steps a to d are performed in one pot.

23. (canceled)

24. (canceled)

25. The method of claim 22, further comprising adding an effective amount of a complexing agent to the peptide in contact with the transition metal catalyst comprising a transition metal, a complex or a salt thereof; wherein the effective amount of the complexing agent is sufficient for substantially complexing the transition metal catalyst; optionally wherein the complexing agent comprises at least one of a thiol, a thiocarbonate, a dithiocarbonate, thiocarbamate, or a dithiocarbamate, including any disulfanyl derivate thereof; optionally wherein the complexing agent comprises glutathione; optionally wherein said transition metal is selected from the group consisting of Pt, Pd, Ru, Cu, Ni, Co, Ti, Zn and Ag or any combination thereof, optionally wherein said transition metal catalyst comprises Pd, a salt or a complex thereof.

26. (canceled)

27. (canceled)

28. (canceled)

29. (canceled)

30. (canceled)

31. The method of claim 22, wherein conditions sufficient for deprotecting said photocleavable protecting group comprises exposing said peptide to a light for a time period sufficient for de-protection of said photocleavable protecting group; wherein said light has a wavelength suitable for deprotecting said photocleavable protecting group; optionally wherein said wavelength is between 200 and 600 nm; optionally wherein said photocleavable protecting group comprises o-nitrobenzyl (NBzl).

32. (canceled)

33. (canceled)

34. The method of claim 21, wherein said reagent is selected from the group consisting of Pd, Cu, sulfide, glutathione, disulfiram (DSF), and diethyldithiocarbamate (DTC), including any combination, any complex or a salt thereof.

35. The method of claim 21, wherein the step b is performed prior to the step c; or wherein the step b and the step c are performed simultaneously.

36. (canceled)

37. (canceled)

38. A composition comprising a peptide comprising one or more intramolecular disulfide bond, and a residual amount of a reagent comprising a disulfide, a diselenide, a thiol, a sulfide, a thiocarbonate, a dithiocarbonate, thiocarbamate, or a dithiocarbamate, including any combination, a disulfanyl derivate, a metal complex or a salt thereof.

39. The composition of claim 38, wherein said reagent comprises any of glutathione, disulfiram (DSF), and diethyldithiocarbamate (DTC), including any combination thereof.

40. (canceled)
